(19) United States
(12) Patent Application Publication
(10) Pub. No.: US 2024/0067923 A1
(43) Pub. Date: Feb. 29, 2024

(54) METHOD FOR PRODUCING BIOLOGIC PRODUCT VARIANTS

(71) Applicant: LONZA LTD, Visp (CH)

(72) Inventor: Rajesh BERI, Westford, MA (US)

(21) Appl. No.: 18/462,800

(22) Filed: Sep. 7, 2023

Related U.S. Application Data
(63) Continuation of application No. 16/486,571, filed on Aug. 16, 2019, now abandoned.
(60) Provisional application No. 62/460,387, filed on Feb. 17, 2017.

(51) Int. Cl. C12N 5/00; A61K 38/02; C12M 1/00

(52) U.S. Cl. CPC C12N 5/0018; A61K 38/02; C12M 29/10; C12M 47/12; C12N 2500/10; C12N 2500/32; C12N 2500/34; C12N 2501/734; C12N 2510/02

(57) ABSTRACT

Disclosed are methods for producing protein variants using multiple culture conditions and perfusion production.

### RELATED APPLICATIONS

This application claims the benefit of U.S. Provisional Application No. 62/460,387, filed Feb. 17, 2017, which is incorporated herein by reference in its entirety.

### FIELD OF THE INVENTION

The present disclosure relates to methods, compositions, and devices for producing biologic product, e.g., protein, e.g., protein variants, using culturing system that operates under a plurality of different culture conditions.

### BACKGROUND

Approved therapeutic products including therapeutic proteins and mAb's are typically only effective in a subset of treated populations. The underlying causes are not always well understood but could be due to the genetic, epigenetic, metabolic, lifestyle and other differences between patients. There is thus an increasing trend to understand the underlying causes of both the disease itself and the patient and develop more tailored therapies. An additional complication now recognized is that the disease itself might vary from patient to patient even though each patient may exhibit the same symptoms of the disease, e.g., it is now recognized that there are 11 different forms of blood cancer (E. Papaermmanuil et. al., N Engl J Med 2016; 374:2209-2221). This increased trend towards precision and personalized treatments is expected to rely on therapeutic product with less variance in product quality attributes. It is also possible that multiple variants of each therapeutic product each with lower heterogeneity (i.e. less variance) in their product quality attributes would be helpful to effectively treat both disease heterogeneity and patient heterogeneity.

Current methods for manufacturing biologic products such as recombinant proteins and monoclonal antibodies (mAbs) typically result in products with a high variability in product quality attributes such as glycosylation, sialyation, and charge variants (Pacis et. al. Biotechnol Bioeng 2011; 108:2348-2358). These methods often rely on fed-batch culture of microbial or animal cells cultured anywhere from 1 to 14 days. The product produced using fed-batch culture is typically a heterogeneous mixture, e.g., the mAb produced by a mammalian cell can have a higher proportion of glycosylated protein chains during the midpoint of the culture (Day 7) than the same mAb produced towards the end of the culture (Day 11 to Day 14). There is no harvesting of product until the end of the culture, thus the product at culture termination is a heterogeneous mixture of product variants. Currently used purification techniques, such as chromatography and filtration steps, are not capable of purifying the product variants and obtaining a more homogeneous product.

### SUMMARY OF THE INVENTION

The present disclosure is based, in part, on the discovery that it is possible to obtain one or both of more product variants and more homogenous preparations of those products by providing a population of cells, culturing the population of cells under a first condition to obtain a first product variant, and recovering first product made under the first condition, then further culturing the population of cells in culture medium under a second condition to obtain a second product variant, and recovering second product made under the second conditions. Thus, a plurality of preparations of different products is made, each product optimized for increased homogeneity.

Accordingly, in one aspect, the invention features a method of making a plurality of variant preparations, the plurality comprising at least a preparation of a first variant of a product (product variant 1) and a preparation of a second variant of a product (product variant 2), comprising: providing a population of cells in a vessel configured to allow cell culture, e.g., perfusion culture;
(a-i) culturing the population of cells in culture medium under a first condition to form conditioned culture medium containing product variant 1;
(a-ii) recovering product variant 1 from culture, e.g., by obtaining an aliquot of conditioned culture medium formed in step (a-i);
(a-iii) optionally adding replacement medium to the conditioned culture medium;
(a-iv) optionally further culturing the population of cells under the first condition to produce additional conditioned medium;
(a-v) optionally recovering additional, e.g., a second batch of, product variant 1, e.g., by obtaining an aliquot of conditioned culture medium formed in step (a-iv);
(a-vi) optionally combining product variant 1 from (a-ii) and (a-v), e.g., from the first and second batches;
(b-i) culturing a population of cells in culture medium under a second condition to form conditioned culture medium containing product variant 2;
(b-ii) recovering product variant 2 from culture, e.g., by obtaining an aliquot of conditioned culture medium formed in step (b-i).
(b-iii) optionally adding replacement medium to the conditioned culture medium,
(b-iv) optionally further culturing the population of cells under the second condition to produce additional conditioned medium.
(b-v) optionally recovering additional, e.g., a second batch of, product variant 2, e.g., by obtaining an aliquot of conditioned culture medium formed in step (b-iv).
(b-vi) optionally combining product variant 2 from (b-ii) and (b-v), e.g., from the first and second batches;
obtaining, e.g., purifying, product variant 1 from a batch of product variant 1;
obtaining, e.g., purifying product variant 2 from a batch of product variant 2;
   thereby providing a plurality of variant preparations, the plurality comprising at least a preparation of a first variant of a product (product variant 1) and a preparation of a second variant of a product (product variant 2),
   wherein variant 1 (or a preparation of variant 1) differs from variant 2 (or a preparation of variant 2) by a physical, chemical, biological, or pharmaceutical property, e.g., by one or more of:
glycosylation (e.g., galactosylation);
sialylation;
charge (e.g., pI);
sequence, e.g., N terminal or C terminal sequence,
homogeneity;
purity;
activity;
amount of inactive variant;
propensity to aggregate, or aggregation;
clarity;
deamidation;
glycation;
proline amidation;
disulfide heterogeneity;
dimerization;
protease susceptibility or proteolytic degradation; and methionine oxidation.

In another aspect, the invention features a method of making a plurality of variant preparations, the plurality comprising at least a preparation of a first variant of a product (product variant 1) and a preparation of a second variant of a product (product variant 2), comprising:
(a) culturing a population of cells in culture medium under a first condition to form conditioned culture medium containing product variant 1;
(b) recovering product variant 1, e.g., a batch of product variant 1, (e.g., produced under the first condition);
(c) culturing the population of cells in culture medium under a second condition to form conditioned culture medium containing product variant 2;
(d) recovering product variant 2, e.g., a batch of product variant 2, (e.g., produced under the second condition);
   thereby providing a plurality of variant preparations, the plurality comprising at least a preparation of a first variant of a product (product variant 1) and a preparation of a second variant of a product (product variant 2),
wherein product variant 1 (or a preparation of product variant 1) differs from product variant 2 (or a preparation of product variant 2) by a physical, chemical, biological, or pharmaceutical property, e.g.,
glycosylation (e.g., galactosylation);
sialylation;
charge (e.g., pI);
sequence, e.g., N terminal or C terminal sequence,
homogeneity;
purity;
activity;
amount of inactive variant;
propensity to aggregate, or aggregation;
clarity;
deamidation;
glycation;
proline amidation;
disulfide heterogeneity;
dimerization;
protease susceptibility or proteolytic degradation; and
methionine oxidation.

In some embodiments, the method comprises, e.g., after obtaining the aliquot of conditioned culture medium, adding replacement medium to the conditioned culture medium.

In some embodiments, the volume of the aliquot removed, the replacement culture medium added, or both, are independently between 5 to 100, 10 to 100, 40 to 100, 60 to 100, 80 to 100, 5 to 10, 5 to 20, 5 to 40, 5 to 60, 5 to 80, 20 to 80, 20 to 60, 20 to 40, or 20 to 80% the volume of the entire culture or of the capacity of the reactor vessel. In some embodiments, the amount removed, the replacement culture medium added, or both, are independently between 0.1 to 5, 0.5 to 5. 0.3 to 5, 0.4 to 5, 0.1 to 4, 0.3 to 4, or 0.5 to 4, 1 to 2, or 1 to 3 times the reactor volume per day of reactor operation.

In some embodiments, the population of cells is cultured under the first condition for 1 or more days (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or more days).

In some embodiments, e.g., after a target value for a parameter is reached, the cell population is cultured under the second condition.

In some embodiments, the method comprises manipulation of the medium or other condition to achieve the second condition.

In some embodiments, the method comprises altering one or more of: pH; level of dO₂; agitation; temperature; volume; density of the cell population; concentration of a component of the culture medium; the presence or amount of a nutrient, drug, inhibitor, or other component.

In some embodiments, components of the culture medium, nutrients, drugs, inhibitors, or other chemical components comprise one or more of media compounds (e.g., PBS, MEM DMEM, serum, etc.), polypeptides, non-peptide signaling molecules (e.g., Ca+, cAMP, glucose, ATP, etc.), amino acids (e.g., lysine), sugars (e.g., galactose or N-acetylmannosamine), and water soluble metal compounds (e.g., copper compounds (e.g., cuprous sulfate or copper chloride), manganese compounds (e.g., manganese chloride), zinc compounds (e.g., zinc chloride), and iron compounds (e.g., ferrous sulfate)).

In some embodiments, the culture of a population of cells is a perfusion production culture, e.g., a perfusion production culture as described herein, e.g., wherein conditioned culture medium is harvested continuously or periodically at set time intervals.

In some embodiments, the method comprises interrupting perfusion as the culture medium in the vessel, e.g., reactor, e.g., bioreactor, transitions to a second condition, e.g., perfusate will not be collected as the culture medium transitions to the second condition and will resume collection upon achieving the second condition, e.g., perfusate will not be collected until product variant 1 is substantially replaced by product variant 2, or as the culture medium transitions to a subsequent condition. In some embodiments, the method comprises diverting perfusate, e.g., to waste, as the culture medium transitions to a second condition, e.g., perfusate will be diverted to waste as the culture medium transitions to the second condition and will resume collection upon achieving the second condition. E.g., perfusate will be diverted to a first destination, e.g., waste, until a first condition is met, e.g., product variant 1 is substantially replaced by product variant 2, or as the culture medium transitions to a subsequent condition.

In some embodiments, product variant 1 is removed from a downstream unit operation, e.g., by flushing with a liquid, e.g., a buffer, e.g., during production of a subsequent product variant, e.g., product variant 2, or after production of a subsequent product variant, e.g., product variant 2.

In some embodiments, the method comprises culturing the cells until a target value for a parameter is reached, e.g., a parameter related to stable operation, e.g., duration of culture, viability of culture, viable cell concentration, pH, dO2, temperature, or volume of culture.

In some embodiments, the population of cells in culture medium of (a), (c), or both (a) and (c) is comprised in a vessel, e.g., a reactor, reactor vessel, or similar. In some embodiments, the population of cells in culture medium of (a) and the population of cells in culture medium of (c) are comprised in the same vessel. In some embodiments, the population of cells in culture medium of (a) and the population of cells in culture medium of (c) are comprised in two or more different vessels.

In some embodiments, the volume of the aliquot removed, the replacement culture medium added, or both, are independently between 5 to 100, 10 to 100, 40 to 100, 60 to 100, 80 to 100, 5 to 10, 5 to 20, 5 to 40, 5 to 60, 5 to 80, 20 to 80, 20 to 60, 20 to 40, or 20 to 80% the volume of the entire culture or of the capacity of the vessel, e.g., reactor. In some embodiments, the amount removed, the replacement culture medium added, or both, are independently between 0.1 to 5, 0.5 to 5. 0.3 to 5, 0.4 to 5, 0.1 to 4, 0.3 to 4, or 0.5 to 4, 1 to 2, or 1 to 3 times the vessel, e.g., reactor, volume per day of vessel, e.g., reactor, operation.

In some embodiments, the volume of replacement culture medium is less than, equal to, or greater than the volume of the aliquot that is removed.

In some embodiments, the volume of the aliquot removed, the replacement culture medium added, or both, are independently between 5 to 100, 10 to 100, 40 to 100, 60 to 100, 80 to 100, 5 to 10, 5 to 20, 5 to 40, 5 to 60, 5 to 80, 20 to 80, 20 to 60, 20 to 40, or 20 to 80% of the volume of the entire culture or of the capacity of the vessel, e.g., reactor. In some embodiments, the amount removed, the replacement culture medium added, or both, are independently between 0.1 to 5, 0.5 to 5. 0.3 to 5, 0.4 to 5, 0.1 to 4, 0.3 to 4, or 0.5 to 4, 1 to 2, or 1 to 3 times the vessel, e.g., reactor, volume per day of vessel, e.g., reactor, operation.

In some embodiments, the population of cells is cultured under the second condition for 1 or more days (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or more days).

In some embodiments, e.g., after a target value for a parameter is reached, the cell population is cultured under a third condition.

In some embodiments, the method comprises interrupting perfusion as the culture medium in the vessel, e.g., reactor, e.g., bioreactor, transitions to a third condition, e.g., perfusate will not be collected as the culture medium transitions to the third condition and will resume collection upon achieving the third condition, e.g., perfusate will not be collected until product variant 2 is substantially replaced by product variant 3, or as the culture medium transitions to a subsequent condition. In some embodiments, the method comprises diverting perfusate to waste as the culture medium transitions to a third condition, e.g., perfusate will be diverted to waste as the culture medium transitions to the third condition and will resume collection upon achieving the third condition, e.g., perfusate will be diverted to waste until product variant 2 is substantially replaced by product variant 3, or as the culture medium transitions to a subsequent condition.

In some embodiments, product variant 2 is removed from a downstream unit operation, e.g., by flushing with a liquid, e.g., a buffer, e.g., during production of a subsequent product variant, e.g., product variant 3, or after production of a subsequent product variant, e.g., product variant 3.

In some embodiments, the plurality comprises a preparation of a third variant made under a third condition, e.g., a preparation of a third variant made under a third condition made by the steps described herein for making the preparation of the first or second variant.

In some embodiments, the plurality comprises a preparation of a fourth variant made under a fourth condition, e.g., a preparation of a fourth variant made under a fourth condition made by the steps described herein for making the preparation of the first or second variant.

In some embodiments, the plurality comprises a preparation of a fifth variant made under a fifth condition, e.g., a preparation of a fifth variant made under a fifth condition made by the steps described herein for making the preparation of the first or second variant.

In some embodiments, the plurality comprises a preparation of an N^{th} variant made under a N^{th} condition, e.g., a preparation of a N^{th} variant made under a N^{th} h condition made by the steps described herein for making the preparation of the first or second variant, wherein N is equal to or greater than 6, 7, 8, 9, 10, 11, 12, 13, 14, 15, 16, 17, 18, 19, 20, 21, 22, 23, 24, 25, 26, 27, 28, 29, or 30.

In some embodiments, step (a) and step (b) are conducted in the same vessel, e.g., a production culture vessel.

In some embodiments, steps (a) through (d) are conducted in the same vessel, e.g., a production culture vessel.

In some embodiments, the vessel is configured to allow operation in perfusion mode.

In some embodiments, the vessel is configured to allow removal of medium and addition of medium during culture, e.g., during one or both of steps (a) and (c).

In some embodiments, the method comprises purifying product variant 1.

In some embodiments, the method comprises purifying product variant 2.

In some embodiments, a product variant is purified in a unit operation downstream from the vessel in which the population of cells is cultured.

In some embodiments, the method comprises providing a plurality of preparations, e.g., a purified preparations, e.g., providing preparations, e.g., a purified preparations, of 2, 3, 4, 5, 6, 7, 8 9 10, 11, 12, 13, 14, 15, 16, 17, 18, 19 or 20 or more different product variants.

In another aspect, the invention features a preparation of a variant product described herein or, made by, or makeable by, any of the methods described herein.

In another aspect, the invention features a plurality of variant preparations, the plurality comprising at least a preparation of a first variant of a product (product variant 1) and a preparation of a second variant of a product (product variant 2), described herein, or made by, or makeable by, any of the methods described herein.

In another aspect, the invention features a vessel, e.g., a bioreactor, e.g., a variable diameter bioreactor, charged with mixture of cells described herein.

In another aspect, the invention features a method of evaluating the progress of a method for making a plurality of product variant preparations, comprising:
(a) culturing a population of cells in culture medium under a first condition to form conditioned culture medium containing a first product variant (product variant 1);
(b) acquiring a value for the progress of the method for making a plurality of product variant preparations toward one or more target parameters selected from: amount of product variant 1 produced, duration of culture under the first condition, or viability of culture;
(c) responsive to the value, determining the progress of the method for making a plurality of product variant preparations toward the one or more target parameters; and
(d) optionally, responsive to the determination that one or more target parameters has been reached, manipulating the culture medium or other condition to achieve a second condition,
thereby evaluating the progress of a method for making a plurality of product variant.

In another aspect, the invention features a method of modifying a method for producing a product variant, comprising:
(a) culturing a population of cells in culture medium under a first condition to form conditioned culture medium containing the product variant (product variant 1);
(b) evaluating the progress of the method for producing a product variant toward one or more target parameters selected from: amount of product variant 1 produced, duration of culture under the first condition, or viability of culture;
(c) responsive to the evaluation of the progress toward the one or more target parameters, manipulating the culture medium or other condition to achieve a second condition; and
(d) optionally, culturing the population of cells in culture medium under the second condition to form conditioned culture medium containing a second product variant (product variant 2),
thereby modifying the method for producing a product variant.

In another aspect, the invention features a pharmaceutical composition comprising a preparation described herein.

In another aspect, the invention features a kit comprising a plurality of variant preparations described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting. Headings, sub-headings or numbered or lettered elements, e.g., (a), (b), (i) etc, are presented merely for ease of reading and are not limiting. The use of headings or numbered or lettered elements in this document does not require the steps or elements be performed in alphabetical order or that the steps or elements are necessarily discrete from one another. Other features, objects, and advantages of the invention will be apparent from the description and drawings, and from the claims.

### BRIEF DESCRIPTION OF THE FIGURES

FIG. 1 is a side view of a variable diameter bioreactor (VDB);

FIG. 2 is a side view of a variable diameter bioreactor (VDB);

FIG. 3 is a side view of a variable diameter bioreactor (VDB);

FIG. 4 is a schematic view of a variable diameter bioreactor (VDB);

FIG. 5 is a schematic view of a variable diameter bioreactor (VDB);

FIG. 6 is a schematic view of a typical bioreactor having a uniform diameter;

FIG. 7 is a schematic view of an example variable diameter bioreactor (VDB);

FIG. 8 is a schematic of an example variable diameter bioreactor (VDB);

FIG. 9 is a schematic of an example variable diameter bioreactor (VDB);

FIG. 10 is a schematic of an example variable diameter bioreactor (VDB); and

FIG. 11 is a schematic of an example variable diameter bioreactor (VDB) bioreactor.

FIG. 12 is a schematic of an example perfusion bioreactor designed as a continuous stirred tank reactor (CSTR).

FIGS. 13A-13C are graphs showing the effects of varying concentration of CuSO₄ (A), Lysine (B), and N-acetylarginine (C) on cell growth, as measured by viable cell density.

FIGS. 14A-14C are graphs showing the effect of lysine (A), CuSO₄ (B), and N-acetylarginine (C) on the charge variance of product (a monoclonal antibody).

FIGS. 15A-15E are graphs showing that increasing CuSO₄ concentration by 2.0 M modulates product charge variance. Shown are changes in percent area for the acidic peak (A), main peak (B), and basic peak (C), as well as the changes between pre- and post-switch charge variance (D and E). *, p<0.05; ***, p<0.0001

FIGS. 16A-16C are graphs showing bidirectional modulation of charge variance in a single perfusion reactor. Shown are changes in percent area for the acidic peak (A), basic peak (B), and mean peak (C) between day 4 and day 62. The timing of switches between nutrient inputs (CuSO₄, basal, or lysine) are indicated.

FIGS. 17A-17E are graphs showing that increasing CuSO₄ concentration by 2.0 M reversibly modulates product charge variance. Shown are changes in percent area for the acidic peak (A), main peak (B), and basic peak (C), as well as the changes in the charge variance between the initial pre-switch basal input condition, during CuSO₄ input, and after the switch back to basal (D). FIG. 17E shows change in the charge variance between basal and during CuSO₄. ****, p<0.0001

FIGS. 18A-18E are graphs showing that increasing the concentration of lysine by 10 mM modulates charge variance. Shown are changes in percent area for the acidic peak (A), main peak (B), and basic peak (C), as well as the changes in charge variance between the basal condition CuSO₄ input and the increased lysine condition (D and E). **, p<0.01; ****, p<0.0001

FIGS. 19A-19D are representative electropherograms showing the effect of the indicated treatments on product charge variance profiles. Shown are the charge variance profiles for the initial basal steady-state condition (day 8; FIG. 19A), the CuSO₄ steady-state condition (day 21; FIG. 19B; arrow indicates should on the basic side of the min peak), the second basal steady-state condition (day 28; FIG. 19C), and the lysine steady-state condition (day 38; FIG. 19D; arrow indicates increase in percent area of the basic peak at an isoelectric point or pI of 7.5),

FIG. 20 is a representative electropherogram showing annotated peak groupings (acidic, main, or basic peaks). The sum of the acidic, main, and basic peaks were calculated to determine the % Area of these groups. Determination of acidic versus basic peaks was based on which side the variants resolved in relation to the main (most abundant, pI 7.2) peak.

FIG. 21 is a graph showing change in percent galactosylation in response to stepwise manipulation of galatose concentration in a perfusion reactor.

FIG. 22 is a graph showing both change in percent galactosylation and reactor galactose concentration in response to stepwise manipulation of galactose concentration in the experiment shown in FIG. 21.

### DETAILED DESCRIPTION OF THE INVENTION

### Definitions

The articles “a” and “an” are used herein to refer to one or to more than one (i.e., to at least one) of the grammatical object of the article. By way of example, “a cell” can mean one cell or more than one cell.

As used herein, the term “aliquot” refers to a volume of a solution, e.g., culture medium or a conditioned culture medium. In an embodiment, each aliquot satisfies a condition with regard to volume, e.g., each aliquot has: a minimal volume, e.g., a preset minimal value; falls within a range between a minimal and a maximal value, e.g., a preset minimal and/or maximal value; approximately equal values, e.g., a preset value; or the same volume, e.g., a preset value. When a larger amount of a liquid, e.g., a conditioned culture medium, is divided into a plurality of aliquots, the plurality may be equal to the entire larger amount, or to less than the entire larger amount. In an embodiment, an aliquot can exceed the volume of a bioreactor, e.g., the culture volume, since aliquots may be removed as replacement culture media is added. In an embodiment, an aliquot is 0.1-5, 0.2-5, 0.3-5, 0.4-5, 0.5-5, 0.5-4, or 0.5-3 culture volumes, wherein culture volume corresponds to the volume of culture in a bioreactor (e.g., a 50 L bioreactor containing a 40 L culture has a culture volume of 40 L, and 0.5 culture volumes of said culture would be 20 L).

As used herein, the term “plurality of aliquots” refers to more than one (e.g., two or more) aliquots.

As used herein, the term “endogenous” refers to any material from or naturally produced inside an organism, cell, tissue or system.

As used herein, the term “exogenous” refers to any material introduced to or produced outside of an organism, cell, tissue or system. Accordingly, “exogenous nucleic acid” refers to a nucleic acid that is introduced to or produced outside of an organism, cell, tissue or system. In an embodiment, sequences of the exogenous nucleic acid are not naturally produced, or cannot be naturally found, inside the organism, cell, tissue, or system that the exogenous nucleic acid is introduced into. Similarly, “exogenous polypeptide” refers to a polypeptide that is not naturally produced, or cannot be naturally found, inside the organism, cell, tissue, or system that the exogenous polypeptide is introduced to, e.g., by expression from an exogenous nucleic acid sequence.

As used herein, the term “heterologous” refers to any material from one species, when introduced to an organism, cell, tissue or system from a different species.

As used herein, the terms “nucleic acid,” “polynucleotide,” or “nucleic acid molecule” are used interchangeably and refers to deoxyribonucleic acid (DNA) or ribonucleic acid (RNA), or a combination of a DNA or RNA thereof, and polymers thereof in either single- or double-stranded form. The term “nucleic acid” includes, but is not limited to, a gene, cDNA, or an mRNA. In one embodiment, the nucleic acid molecule is synthetic (e.g., chemically synthesized or artificial) or recombinant. Unless specifically limited, the term encompasses molecules containing analogues or derivatives of natural nucleotides that have similar binding properties as the reference nucleic acid and are metabolized in a manner similar to naturally or non-naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

As used herein, the terms “peptide,” “polypeptide,” and “protein” are used interchangeably, and refer to a compound comprised of amino acid residues covalently linked by peptide bonds, or by means other than peptide bonds. A protein or peptide must contain at least two amino acids, and no limitation is placed on the maximum number of amino acids that can comprise a protein's or peptide's sequence. In one embodiment, a protein may comprise of more than one, e.g., two, three, four, five, or more, polypeptides, in which each polypeptide is associated to another by either covalent or non-covalent bonds/interactions. Polypeptides include any peptide or protein comprising two or more amino acids joined to each other by peptide bonds or by means other than peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others.

“Product” as that term is used herein refers to a molecule, e.g., polypeptide, e.g., protein, e.g., glycoprotein, nucleic acid, lipid, saccharade, polysaccharide, or any hybrid thereof, that is produced, e.g., expressed, by a cell, e.g., a cell which has been modified or engineered to produce the product. In one embodiment, the product comprises a naturally occurring product. In an embodiment the product comprises a non-naturally occurring product. In one embodiment, a portion of the product is naturally occurring, while another portion of the product is non-naturally occurring. In one embodiment, the product is a polypeptide, e.g., a recombinant polypeptide. In one embodiment, the product is suitable for diagnostic or pre-clinical use. In another embodiment, the product is suitable for therapeutic use, e.g., for treatment of a disease. In one embodiment, cells described herein, e.g., modified or engineered cells, comprise an exogenous nucleic acid that controls expression or encodes the product. In other embodiments, cells described herein, e.g., modified or engineered cells, comprise other molecules, e.g., that are not nucleic acids, that controls the expression or construction of the product in the cell.

As used herein, “variant of a product,” “variant,” “product variant,” or similar term refers to a species of product which differs from a reference product. E.g., a first product made under a first set of conditions, having a structural or functional property that differs from a second product made under a second set of conditions, are variants. Typically, the variants are expressed from the same cell(s) or from the same encoding sequence. For example, a first variant of a product may be glycosylated, whereas a second variant of a product may be differently glycosylated (e.g., glycosylated to a greater or lesser extent, or appended with at least one different sugar moiety). Properties distinguishing product variants include physical, chemical, biological, or pharmaceutical properties, and include, but are not limited to: glycosylation (e.g., galactosylation), sialylation, charge (e.g., pI), sequence (e.g., N terminal or C terminal sequence), therapeutic efficacy, propensity to aggregate or propensity of aggregation, or activity. Properties distinguishing product variants are also called product quality attributes. A product variant that differs from a reference product with respect to a particular product quality attribute may be referred to as such (e.g., a product variant that differs with respect to charge (e.g., pI) may be referred to as a charge variant). Variants can also differ by “preparation” or bulk properties, e.g., a preparation of a first product variant can differ from a second product variant in homogeneity, purity, amount of aggregration, amount of inactive variant, clarity, or shelf life.

As used herein, the terms “plurality of variants”, “plurality of variant preparations”, “plurality of product variants” and similar refer to more than one (e.g., two or more) variants, variant preparations, product variants, etc.

As used herein, “condition” refers to a value of one or more culture or environmental parameters that can influence growth and/or gene expression in a culture of cells. A first condition may be conducive to expression of a first product variant, e.g., forming conditioned culture medium containing a first product variant; whereas a second condition may be conducive to expression of a second product variant, e.g., forming conditioned culture medium containing a second product variant. Culture or environmental parameters include, but are not limited to, medium type (e.g., PBS, MEM DMEM, serum, serum containing media, etc.), the levels of one or more polypeptides, chemical salts, metal and metal ions, amino acids, amino acid derivatives, sugars composition, hexosamines, n-acetylhexosamines, vitamins, lipids, polyamines, reducing/oxidizing agents, the level of non-peptide signaling molecules (e.g., Ca+², cAMP, glucose, ATP, etc.), temperature, pH, cell density of culture, and nutrient availability. A steady-state condition (also referred to as a steady-state or steady state) refers to a condition where cell density and one or more product quality attributes remain constant.

### Biologic Production Methodologies

In some embodiments, biologics such as recombinant proteins and mAb's are produced in batch, fed-batch, or perfusion cultures of microbial (e.g. E. coli), animal (e.g., mammalian (e.g., CHO or NSO), fungal (e.g., Pichia pastoris) or insect), or plant cells. The population of cells used to produce a product and the culture medium the population is grown in is the production culture.

In some embodiments, production is initiated by culturing a small population of previously frozen cells in a medium comprising carbohydrates, amino acids, proteins, lipids, vitamins, nucleosides, and/or chemical salts under controlled conditions e.g. temperature, pH, dissolved oxygen and agitation. The culture is expanded under these conditions in increasingly larger volumes until a sufficient population of cells is generated and production of therapeutic product protein can be initiated in production cultures which typically range in volume from 50 L to 20,000 liters. The duration of this culture group phase can last from a few hours (microbial culture) to about 30 days for animal cell cultures.

There are currently two main approaches to producing product; batch or fed batch production and perfusion production. In batch or fed-batch production, the entire production culture is harvested on a specified day usually anywhere from 1 day to 21 days from initiation. In batch production, all the nutrients and substrates required for production are present in the culture medium from the beginning of culturing, whereas in fed batch production nutrients and/or substrates are added or fed into the culture during culturing.

In perfusion production culture, the production culture is harvested continuously or at specified intervals over a period of time. In an embodiment, about 0.5 to 3 culture volumes is harvested daily. Product variant can be purified from harvested culture volumes. In some embodiments, the production culture is replenished with replacement, e.g., fresh, culture medium continuously or at intervals over a period of time. In an embodiment, the culture is replaced by an equivalent volume of fresh medium. In some embodiments, the culture duration can last anywhere from 30 days to 150 days (e.g., 40, 50, 60, 70, 80, 90, 100, 110, 120, 130, or 140 days) post culture initiation.

### Production of Homogeneous Product Variants by Perfusion Culture

Disclosed herein are methods of preparation of one or more products by use of a culture system, e.g., a perfusion production culture system, to produce one or more product variants. In an embodiment a preparation of product variant has optimized homogeneity with respect to a property, e.g., glycosylation, activity, or homogeneity, e.g., as compared to a product produced by a batch or fed-batch production culture. While not wishing to be bound by theory, it is believed that the heterogeneity of product produced from fed-batch and batch production cultures stems from the preparation comprising product variants made over a period of time encompassing significantly different culture conditions, e.g., the entire duration of the production culture. Thus more than one form of the product is present in the final harvested culture medium. The smaller, periodic aliquots harvested from systems described herein comprise product variants with increased homogeneity, possibly due to the potentially more homogeneous cellular and environmental conditions the product variants from perfusion cultures were produced under.

Disclosed herein are methods of preparation of two or more products by use of a culture system described herein, e.g., a perfusion production culture system, to produce two or more preparations of product variants, each with optimized homogeneity, e.g., increased homogeneity with respect to a property as compared to a product produced by a batch or fed-batch production culture. In some embodiments, a first product variant is produced by culturing the production culture under a first condition. Batches (e.g., one or more batches) of product recovered from the production culture under the first condition comprise the first product variant. A second product variant is produced by culturing the production culture under a second condition. Batches (e.g., one or more batches) of product recovered from the production culture under the second condition would comprise the second product variant.

In some embodiments, a condition comprises culture and/or environmental parameters including but not limited to medium type (e.g., PBS, MEM DMEM, serum, serum containing media, etc.), the levels of one or more polypeptides, chemical salts, metal and metal ions, amino acids, amino acid derivatives, sugars composition, hexosamines, n-acetylhexosamines, vitamins, lipids, polyamines, reducing/oxidizing agents, the level of non-peptide signaling molecules (e.g., Ca⁺², cAMP, glucose, ATP, etc.), temperature, pH, cell density of culture, and nutrient availability. In some embodiments, a condition comprises levels (e.g., concentrations or relative levels, e.g., increased or decreased levels relative to a previous condition) of one or more of amino acids (e.g., lysine), sugars (e.g., galactose or N-acetylmannosamine), or water soluble metal compounds. Amino acids and sugars for use in creating or maintaining a condition may comprise a variety of covalent modifications (e.g., acetylation). Water soluble metal compounds useful in creating or maintaining conditions include but are not limited to copper compounds (e.g., cuprous sulfate or copper chloride), manganese compounds (e.g., manganese chloride), zinc compounds (e.g., zinc chloride), and iron compounds (e.g., ferrous sulfate).

In some embodiments, the methods of preparation of the invention produce more than two preparations of product, e.g., more than two product variants. In some embodiments, the culture can be further cultured under a third, fourth, fifth, or further condition. Batches (e.g., one or more batches) of product recovered from the production culture under the third, fourth, fifth, or further condition would comprise the third, fourth, fifth, or further product variant.

In an embodiment, the method provides a preparation of a first variant and a preparation of a second variant. In an embodiment the method provides a preparation of a third variant.

In an embodiment the method provides a preparation of a fourth variant.

In an embodiment the method provides a preparation of a fifth variant.

In an embodiment the method provides a preparation of a sixth variant.

In an embodiment the method provides a preparation of a seventh variant.

In an embodiment the method provides a preparation of a eighth variant.

In an embodiment the method provides a preparation of a ninth variant.

In an embodiment the method provides a preparation of a tenth variant.

In an embodiment the method provides a preparation of a eleventh variant.

In an embodiment the method provides a preparation of a twelfth variant.

In an embodiment the method provides a preparation of a thirteenth variant.

In an embodiment the method provides a preparation of a fourteenth variant.

In an embodiment the method provides a preparation of a fifteenth variant.

In an embodiment the method provides a preparation of a sixteenth variant.

In an embodiment the method provides a preparation of a seventeenth variant.

In an embodiment the method provides a preparation of a eighteenth variant.

In an embodiment the method provides a preparation of a nineteenth variant.

In an embodiment the method provides a preparation of a twentieth variant.

In some embodiments, after desired production of a product variant is complete, the production culture is cultured under a next condition. In some embodiments, after the transition to the next condition, components of the bioreactor or components downstream of the bioreactor are cleansed of the former product variant. In some embodiments, during this cleansing period, perfusate is not collected or is collected and discarded. Once the bioreactor has reached stable operation, collection and purification of perfusate, e.g., perfusate comprising the next product variant, may resume.

In an embodiment one or a plurality of product variants, or preparations thereof, are analyzed, e.g., for the presence, e.g., level, of a parameter, e.g., glycosylation, that differs between the variants.

### Applications For Production

The methods of preparation of products, e.g., product variants, disclosed herein can be used to produce a variety of products, evaluate various cell lines, or to evaluate the production of various cell lines for use in a bioreactor or processing vessel or tank, or, more generally with any feed source. The devices, facilities and methods described herein are suitable for culturing any desired cell line including prokaryotic and/or eukaryotic cell lines. Further, in embodiments, the devices, facilities and methods are suitable for culturing suspension cells or anchorage-dependent (adherent) cells and are suitable for production operations configured for production of pharmaceutical and biopharmaceutical products-such as polypeptide products, nucleic acid products (for example DNA or RNA), or cells and/or viruses such as those used in cellular and/or viral therapies.

In embodiments, the cells express or produce a product, such as a recombinant therapeutic or diagnostic product. As described in more detail below, examples of products produced by cells include, but are not limited to, antibody molecules (e.g., monoclonal antibodies, bispecific antibodies), antibody mimetics (polypeptide molecules that bind specifically to antigens but that are not structurally related to antibodies such as e.g. DARPins, affibodies, adnectins, or IgNARs), fusion proteins (e.g., Fc fusion proteins, chimeric cytokines), other recombinant proteins (e.g., glycosylated proteins, enzymes, hormones), viral therapeutics (e.g., anti-cancer oncolytic viruses, viral vectors for gene therapy and viral immunotherapy), cell therapeutics (e.g., pluripotent stem cells, mesenchymal stem cells and adult stem cells), vaccines or lipid-encapsulated particles (e.g., exosomes, virus-like particles), RNA (such as e.g. siRNA) or DNA (such as e.g. plasmid DNA), antibiotics or amino acids. In embodiments, the devices, facilities and methods can be used for producing biosimilars.

As mentioned, in embodiments, devices, facilities and methods allow for the production of eukaryotic cells, e.g., mammalian cells or lower eukaryotic cells such as for example yeast cells or filamentous fungi cells, or prokaryotic cells such as Gram-positive or Gram-negative cells and/or products of the eukaryotic or prokaryotic cells, e.g., proteins, peptides, antibiotics, amino acids, nucleic acids (such as DNA or RNA), synthesised by the eukaryotic cells in a large-scale manner. Unless stated otherwise herein, the devices, facilities, and methods can include any desired volume or production capacity including but not limited to bench-scale, pilot-scale, and full production scale capacities.

Moreover and unless stated otherwise herein, the devices, facilities, and methods can include any suitable reactor(s) including but not limited to stirred tank, airlift, fiber, microfiber, hollow fiber, ceramic matrix, fluidized bed, fixed bed, and/or spouted bed bioreactors. As used herein, “reactor” can include a fermentor or fermentation unit, or any other reaction vessel and the term “reactor” is used interchangeably with “fermentor.” For example, in some aspects, a bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and C02 levels, maintenance of pH level, agitation (e.g., stirring), and/or cleaning/sterilizing. Example reactor units, such as a fermentation unit, may contain multiple reactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple reactors within the facility. In various embodiments, the bioreactor can be suitable for batch, semi fed-batch, fed-batch, perfusion, and/or a continuous fermentation processes. Any suitable reactor diameter can be used. In embodiments, the bioreactor can have a volume between about 100 mL and about 50,000 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Additionally, suitable reactors can be multi-use, single-use, disposable, or non-disposable and can be formed of any suitable material including metal alloys such as stainless steel (e.g., 316L or any other suitable stainless steel) and Inconel, plastics, and/or glass.

In embodiments and unless stated otherwise herein, the devices, facilities, and methods described herein for use with methods of making a preparation can also include any suitable unit operation and/or equipment not otherwise mentioned, such as operations and/or equipment for separation, purification, and isolation of such products. Any suitable facility and environment can be used, such as traditional stick-built facilities, modular, mobile and temporary facilities, or any other suitable construction, facility, and/or layout. For example, in some embodiments modular clean-rooms can be used. Additionally and unless otherwise stated, the devices, systems, and methods described herein can be housed and/or performed in a single location or facility or alternatively be housed and/or performed at separate or multiple locations and/or facilities.

By way of non-limiting examples and without limitation, U.S. Publication Nos. 2013/0280797; 2012/0077429; 2011/0280797; 2009/0305626; and U.S. Pat. Nos. 8,298,054; 7,629,167; and 5,656,491, which are hereby incorporated by reference in their entirety, describe example facilities, equipment, and/or systems that may be suitable.

Methods of making a preparation described herein can use a broad spectrum of cells. In embodiments, the cells are eukaryotic cells, e.g., mammalian cells. The mammalian cells can be for example human or rodent or bovine cell lines or cell strains. Examples of such cells, cell lines or cell strains are e.g. mouse myeloma (NSO)-cell lines, Chinese hamster ovary (CHO)-cell lines, HT1080, H9, HepG2, MCF7, MDBK Jurkat, NIH3T3, PC12, BHK (baby hamster kidney cell), VERO, SP2/0, YB2/0, Y0, C127, L cell, COS, e.g., COS1 and COS7, QC1-3,HEK-293, VERO, PER.C6, HeLA, EB1, EB2, EB3, oncolytic or hybridoma-cell lines. Preferably the mammalian cells are CHO-cell lines. In one embodiment, the cell is a CHO cell. In one embodiment, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1 SV GS knockout cell. The CHO FUT8 knockout cell is, for example, the Potelligent® CHOK1 SV (Lonza Biologics, Inc.). Eukaryotic cells can also be avian cells, cell lines or cell strains, such as for example, EBx® cells, EB14, EB24, EB26, EB66, or EBv13.

In one embodiment, the eukaryotic cells are stem cells. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs).

In one embodiment, the cell is a differentiated form of any of the cells described herein. In one embodiment, the cell is a cell derived from any primary cell in culture.

In embodiments, the cell is a hepatocyte such as a human hepatocyte, animal hepatocyte, or a non-parenchymal cell. For example, the cell can be a plateable metabolism qualified human hepatocyte, a plateable induction qualified human hepatocyte, plateable Qualyst Transporter Certified™ human hepatocyte, suspension qualified human hepatocyte (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C57BI/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes). Example hepatocytes are commercially available from Triangle Research Labs, LLC, 6 Davis Drive Research Triangle Park, North Carolina, USA 27709.

In one embodiment, the eukaryotic cell is a lower eukaryotic cell such as e.g. a yeast cell (e.g., Pichia genus (e.g. Pichia pastoris, Pichia methanolica, Pichia kluyveri, and Pichia angusta), Komagataella genus (e.g. Komagataella pastoris, Komagataella pseudopastoris or Komagataella phaffii), Saccharomyces genus (e.g. Saccharomyces cerevisae, cerevisiae, Saccharomyces kluyveri, Saccharomyces uvarum), Kluyveromyces genus (e.g. Kluyveromyces lactis, Kluyveromyces marxianus), the Candida genus (e.g. Candida utilis, Candida cacaoi, Candida boidinii), the Geotrichum genus (e.g. Geotrichum fermentans), Hansenula polymorpha, Yarrowia lipolytica, or Schizosaccharomyces pombe. Preferred is the species Pichia pastoris. Examples for Pichia pastoris strains are X33, GS115, KM71, KM71H; and CBS7435.

In one embodiment, the eukaryotic cell is a fungal cell (e.g. Aspergillus (such as A. niger, A. fumigatus, A. orzyae, A. nidula), Acremonium (such as A. thermophilum), Chaetomium (such as C. thermophilum), Chrysosporium (such as C. thermophile), Cordyceps (such as C. militaris), Corynascus, Ctenomyces, Fusarium (such as F. oxysporum), Glomerella (such as G. graminicola), Hypocrea (such as H. jecorina), Magnaporthe (such as M. orzyae), Myceliophthora (such as M. thermophile), Nectria (such as N. heamatococca), Neurospora (such as N. crassa), Penicillium, Sporotrichum (such as S. thermophile), Thielavia (such as T. terrestris, T. heterothallica), Trichoderma (such as T. reesei), or Verticillium (such as V. dahlia)).

In one embodiment, the eukaryotic cell is an insect cell (e.g., Sf9, Mimic™ Sf9, Sf21, High Five™ (BT1-TN-5B1-4), or BT1-Ea88 cells), an algae cell (e.g., of the genus Amphora, Bacillariophyceae, Dunaliella, Chlorella, Chlamydomonas, Cyanophyta (cyanobacteria), Nannochloropsis, Spirulina, or Ochromonas), or a plant cell (e.g., cells from monocotyledonous plants (e.g., maize, rice, wheat, or Setaria), or from a dicotyledonous plants (e.g., cassava, potato, soybean, tomato, tobacco, alfalfa, Physcomitrella patens or Arabidopsis).

In one embodiment, the cell is a bacterial or prokaryotic cell.

In embodiments, the prokaryotic cell is a Gram-positive cells such as Bacillus, Streptomyces Streptococcus, Staphylococcus or Lactobacillus. Bacillus that can be used is, e.g. the B.subtilis, B.amyloliquefaciens, B.licheniformis, B. natto, or B.megaterium. In embodiments, the cell is B.subtilis, such as B.subtilis 3NA and B.subtilis 168. Bacillus is obtainable from, e.g., the Bacillus Genetic Stock Center, Biological Sciences 556, 484 West 12^{t}h Avenue, Columbus OH 43210-1214.

In one embodiment, the prokaryotic cell is a Gram-negative cell, such as Salmonella spp. orEscherichia coli, such as e.g., TG1, TG2, W3110, DH1, DHB4, DH5a, HMS 174, HMS174 (DE3), NM533, C600, HB101, JM109, MC4100, XL1-Blue and Origami, as well as those derived from E. coli B-strains, such as for example BL-21 or BL21 (DE3), all of which are commercially available.

Suitable host cells are commercially available, for example, from culture collections such as the DSMZ (Deutsche Sammlung von Mikroorganismen and Zellkulturen GmbH, Braunschweig, Germany) or the American Type Culture Collection (ATCC).

In embodiments, the cultured cells are used to produce proteins e.g., antibodies, e.g., monoclonal antibodies, and/or recombinant proteins, for therapeutic use. In embodiments, the cultured cells produce peptides, amino acids, fatty acids or other useful biochemical intermediates or metabolites. For example, in embodiments, molecules having a molecular weight of about 4000 daltons to greater than about 140,000 daltons can be produced. In embodiments, these molecules can have a range of complexity and can include posttranslational modifications including glycosylation.

In embodiments, the polypeptide is, e.g., BOTOX, Myobloc, Neurobloc, Dysport (or other serotypes of botulinum neurotoxins), alglucosidase alpha, daptomycin, YH-16, choriogonadotropin alpha, filgrastim, cetrorelix, interleukin-2, aldesleukin, teceleulin, denileukin diftitox, interferon alpha-n3 (injection), interferon alpha-nl, DL-8234, interferon, Suntory (gamma-1a), interferon gamma, thymosin alpha 1, tasonermin, DigiFab, ViperaTAb, EchiTAb, CroFab, nesiritide, abatacept, alefacept, Rebif, eptoterminalfa, teriparatide, calcitonin, etanercept, hemoglobin glutamer 250 (bovine), drotrecogin alpha, collagenase, carperitide, recombinant human epidermal growth factor, DWP401, darbepoetin alpha, epoetin omega, epoetin beta, epoetin alpha, desirudin, lepirudin, bivalirudin, nonacog alpha, Mononine, eptacog alpha (activated), recombinant Factor VIII+VWF, Recombinate, recombinant Factor VIII, Factor VIII (recombinant), Alphnmate, octocog alpha, Factor VIII, palifermin,Indikinase, tenecteplase, alteplase, pamiteplase, reteplase, nateplase, monteplase, follitropin alpha, rFSH, hpFSH, micafungin, pegfilgrastim, lenograstim, nartograstim, sermorelin, glucagon, exenatide, pramlintide, iniglucerase, galsulfase, Leucotropin, molgramostirn, triptorelin acetate, histrelin (Hydron), deslorelin, histrelin, nafarelin, leuprolide (ATRIGEL), leuprolide (DUROS), goserelin, Eutropin, somatropin, mecasermin, enlfavirtide, Org-33408, insulin glargine, insulin glulisine, insulin (inhaled), insulin lispro, insulin deternir, insulin (RapidMist), mecasermin rinfabate, anakinra, celmoleukin, 99 mTc-apcitide, myelopid, Betaseron, glatiramer acetate, Gepon, sargramostim, oprelvekin, human leukocyte-derived alpha interferons, Bilive, insulin (recombinant), recombinant human insulin, insulin aspart, mecasenin, Roferon-A, interferon-alpha 2, Alfaferone, interferon alfacon-1, interferon alpha, Avonex’ recombinant human luteinizing hormone, dornase alpha, trafermin, ziconotide, taltirelin, diboterminalfa, atosiban, becaplermin, eptifibatide, Zemaira, CTC-111, Shanvac-B, octreotide, lanreotide, ancestirn, agalsidase beta, agalsidase alpha, laronidase, prezatide copper acetate, rasburicase, ranibizumab, Actimmune, PEG-Intron, Tricomin, recombinant human parathyroid hormone (PTH) 1-84, epoetin delta, transgenic antithrombin III, Granditropin, Vitrase, recombinant insulin, interferon-alpha, GEM-21S, vapreotide, idursulfase, omnapatrilat, recombinant serum albumin, certolizumab pegol, glucarpidase, human recombinant C1 esterase inhibitor, lanoteplase, recombinant human growth hormone, enfuvirtide, VGV-1, interferon (alpha), lucinactant, aviptadil, icatibant, ecallantide, omiganan, Aurograb, pexigananacetate, ADI-PEG-20, LDI-200, degarelix, cintredelinbesudotox, Favld, MDX-1379, ISAtx-247, liraglutide, teriparatide, tifacogin, AA4500, T4N5 liposome lotion, catumaxomab, DWP413, ART-123, Chrysalin, desmoteplase, amediplase, corifollitropinalpha, TH-9507, teduglutide, Diamyd, DWP-412, growth hormone, recombinant G-CSF, insulin, insulin (Technosphere), insulin (AERx), RGN-303, DiaPep277, interferon beta, interferon alpha-n3, belatacept, transdermal insulin patches, AMG-531, MBP-8298, Xerecept, opebacan, AIDSVAX, GV-1001, LymphoScan, ranpirnase, Lipoxysan, lusupultide, MP52, sipuleucel-T, CTP-37, Insegia, vitespen, human thrombin, thrombin, TransMID, alfimeprase, Puricase, terlipressin, EUR-1008M, recombinant FGF-I, BDM-E, rotigaptide, ETC-216, P-113, MBI-594AN, duramycin, SCV-07, OPI-45, Endostatin, Angiostatin, ABT-510, Bowman Birk Inhibitor, XMP-629, 99 mTc-Hynic-Annexin V, kahalalide F, CTCE-9908, teverelix, ozarelix, rornidepsin, BAY-504798, interleukin4, PRX-321, Pepscan, iboctadekin, rhlactoferrin, TRU-015, IL-21, ATN-161, cilengitide, Albuferon, Biphasix, IRX-2, omega interferon, PCK-3145, CAP-232, pasireotide, huN901-DMI, SB-249553, Oncovax-CL, OncoVax-P, BLP-25, CerVax-16, MART-1, gp100, tyrosinase, nemifitide, rAAT, CGRP, pegsunercept, thymosinbeta4, plitidepsin, GTP-200, ramoplanin, GRASPA, OBI-1, AC-100, salmon calcitonin (eligen), examorelin, capromorelin, Cardeva, velafermin, 131I-TM-601, KK-220, T-10, ularitide, depelestat, hematide, Chrysalin, rNAPc2, recombinant Factor V111 (PEGylated liposomal), bFGF, PEGylated recombinant staphylokinase variant, V-10153, SonoLysis Prolyse, NeuroVax, CZEN-002, rGLP-1, BIM-51077, LY-548806, exenatide (controlled release, Medisorb), AVE-0010, GA-GCB, avorelin, ACM-9604, linaclotid eacetate, CETi-1, Hemospan, VAL, fast-acting insulin (injectable, Viadel), insulin (eligen), recombinant methionyl human leptin, pitrakinra, Multikine, RG-1068, MM-093, NBI-6024, AT-001, PI-0824, Org-39141, Cpn10, talactoferrin, rEV-131, rEV-131, recombinant human insulin, RPI-78M, oprelvekin, CYT-99007 CTLA4-Ig, DTY-001, valategrast, interferon alpha-n3, IRX-3, RDP-58, Tauferon, bile salt stimulated lipase, Merispase, alaline phosphatase, EP-2104R, Melanotan-II, bremelanotide, ATL-104, recombinant human microplasmin, AX-200, SEMAX, ACV-1, Xen-2174, CJC-1008, dynorphin A, SI-6603, LAB GHRH, AER-002, BGC-728, ALTU-135, recombinant neuraminidase, Vacc-5q, Vacc-4x, Tat Toxoid, YSPSL, CHS-13340, PTH(1-34) (Novasome), Ostabolin-C, PTH analog, MBRI-93.02, MTB72F, MVA-Ag85A, FARA04, BA-210, recombinant plague FIV, AG-702, OxSODrol, rBetV1, Der-p1/Der-p2/Der-p7, PR1 peptide antigen, mutant ras vaccine, HPV-16 E7 lipopeptide vaccine, labyrinthin, WT1-peptide, IDD-5, CDX-110, Pentrys, Norelin, CytoFab, P-9808, VT-111, icrocaptide, telbermin, rupintrivir, reticulose, rGRF, HA, alpha-galactosidase A, ACE-011, ALTU-140, CGX-1160, angiotensin, D-4F, ETC-642, APP-018, rhMBL, SCV-07, DRF-7295, ABT-828, ErbB2-specific immunotoxin, DT3SSIL-3, TST-10088, PRO-1762, Combotox, cholecystokinin-B/gastrin-receptor binding peptides, 111In-hEGF, AE-37, trasnizumab-DM1, Antagonist G, IL-12, PM-02734, IMP-321, rhIGF-BP3, BLX-883, CUV-1647, L-19 based ra, Re-188-P-2045, AMG-386, DC/1540/KLH, VX-001, AVE-9633, AC-9301, NY-ESO-1 (peptides), NA17.A2 peptides, CBP-501, recombinant human lactoferrin, FX-06, AP-214, WAP-8294A, ACP—HIP, SUN-11031, peptide YY [3-36], FGLL, atacicept, BR3-Fc, BN-003, BA-058, human parathyroid hormone 1-34, F-18-CCR1, AT-1100, JPD-003, PTH(7-34) (Novasome), duramycin, CAB-2, CTCE-0214, GlycoPEGylated erythropoietin, EPO-Fc, CNTO-528, AMG-114, JR-013, Factor XIII, aminocandin, PN-951, 716155, SUN-E7001, TH-0318, BAY-73-7977, teverelix, EP-51216, hGH, OGP-I, sifuvirtide, TV4710, ALG-889, Org-41259, rhCC10, F-991, thymopentin, r(m)CRP, hepatoselective insulin, subalin, L19-IL-2 fusion protein, elafin, NMK-150, ALTU-139, EN-122004, rhTPO, thrombopoietin receptor agonist, AL-108, AL-208, nerve growth factor antagonists, SLV-317, CGX-1007, INNO-105, teriparatide (eligen), GEM-OS1, AC-162352, PRX-302, LFn-p24 fusion, EP-1043, gpE1, gpE2, MF-59, hPTH(1-34), 768974, SYN-101, PGN-0052, aviscumnine, BIM-23190, multi-epitope tyrosinase peptide, enkastim, APC-8024, GI-5005, ACC-001, TTS-CD3, vascular-targeted TNF, desmopressin, onercept, and TP-9201.

In some embodiments, the polypeptide is adalimumab (HUMIRA), infliximab (REMICADE™), rituximab (RITUXAN™/MAB THERA™) etanercept (ENBREL™) bevacizumab (AVASTIN™), trastuzumab (HERCEPTIN™), pegrilgrastim (NEULASTA™), or any other suitable polypeptide including biosimilars and biobetters.

Other suitable polypeptides are those listed below and in Table 1 of US2016/0097074:

TABLE 1

| Protein Product | Reference Listed Drug |
|---|---|
| interferon gamma-1b | Actimmune ® |
| alteplase; tissue plasminogen activator | Activase ®/Cathflo ® |
| Recombinant antihemophilic factor | Advate |
| human albumin | Albutein ® |
| Laronidase | Aldurazyme ® |
| Interferon alfa-N3, human leukocyte derived | Alferon N ® |
| human antihemophilic factor | Alphanate ® |
| virus-filtered human coagulation factor IX | AlphaNine ® SD |
| Alefacept: recombinant, dimeric fusion protein LFA3-Ig | Amevive ® |
| Bivalirudin | Angiomax ® |
| darbepoetin alfa | Aranesp ™ |
| Bevacizumab | Avastin ™ |
| interferon beta-1a; recombinant | Avonex ® |
| coagulation factor IX | BeneFix ™ |
| Interferon beta-1b | Betaseron ® |
| Tositumomab | BEXXAR ® |
| antihemophilic factor | Bloclate ™ |
| human growth hormone | BioTropin ™ |
| botulinum toxin type A | BOTOX ® |
| Alemtuzumab | Campath ® |
| acritumomab; technetium-99 labeled | CEA-Scan ® |
| alglucerase; modified form of beta-glucocerebrosidase | Ceredase ® |
| imiglucerase; recombinant form of beta-glucocerebrosidase | Cerezyme ® |
| crotalidae polyvalent immune Fab, ovine | CroFab ™ |
| digoxin immune fab [ovine] | Digifab ™ |
| Rasburicase | Elitek ® |
| Etanercept | ENBREL ® |
| epoietin alfa | Epogen ® |
| Cetuximab | Erbitux ™ |
| algasidase beta | Fabrazyme ® |
| Urofollitropin | Fertinex ™ |
| follitropin beta | Follistim ™ |
| Teriparatide | FORTEO ® |
| human somatropin | GenoTropin ® |
| Glucagon | GlucaGen ® |
| follitropin alfa | Gonal-F ® |
| antihemophilic factor | Helixate ® |
| Antihemophilic Factor; Factor XIII | HEMOFIL |
| adefovir dipivoxil | Hepsera ™ |
| Trastuzumab | Herceptin ® |
| Insulin | Humalog ® |
| antihemophilic factor/von Willebrand factor complex-human | Humate-P ® |
| Somatotropin | Humatrope ® |
| Adalimumab | HUMIRA ™ |
| human insulin | Humulin ® |
| recombinant human hyaluronidase | Hylenex ™ |
| interferon alfacon-1 | Infergen ® |
| eptifibatide | Integrilin ™ |
| alpha-interferon | Intron A ® |
| Palifermin | Kepivance |
| Anakinra | Kineret ™ |
| antihemophilic factor | Kogenate ® FS |
| insulin glargine | Lantus ® |
| granulocyte macrophage colony-stimulating factor | Leukine ®/Leukine ® Liquid |
| lutropin alfa for injection | Luveris |
| OsoA lipoprotein | LYMErix ™ |
| Ranibizumab | LUCENTIS ® |
| gemtuzumab ozogamicin | Mylotarg ™ |
| Galsulfase | Naglazyme ™ |
| Nesiritide | Natrecor ® |
| Pegfilgrastim | Neulasta ™ |
| Oprelvekin | Neumega ® |
| Filgrastim | Neupogen ® |
| Fanolesomab | NeutroSpec ™ (formerly LeuTech ®) |
| somatropin [rDNA] | Norditropin ®/Norditropin Nordiflex ® |
| Mitoxantrone | Novantrone ® |
| insulin; zinc suspension; | Novolin L ® |
| insulin; isophane suspension | Novolin N ® |
| insulin, regular; | Novolin R ® |
| Insulin | Novolin ® |
| coagulation factor VIIa | NovoSeven ® |
| Somatropin | Nutropin ® |
| immunoglobulin intravenous | Octagam ® |
| PEG-L-asparaginase | Oncaspar ® |
| abatacept, fully human soluable fusion protein | Orencia ™ |
| muromomab-CD3 | Orthoclone OKT3 ® |
| high-molecular weight hyaluronan | Orthovisc ® |
| human chorionic gonadotropin | Ovidrel ® |
| live attenuated Bacillus Calmette-Guerin | Pacis ® |
| abatacept, fully human soluable protein | Orencia ™ |
| muromomab-CD3 | Orthocione OKT3 ® |
| high-molecular weight hyaluronan | Orthovisc ® |
| human chorlonic gonadotropin | Ovidrel ® |
| live attenuated Bacillus Calmette-Guerin | Pacis ® |
| peginterferon alfa-2a | Pegasys ® |
| pegylated version of interferon alfa-2b | PEG-Intron ™ |
| Abarelix (injectable suspension); gonadotropin-releasing hormone | Plenaxis ™ |
| antagonist | |
| epoietin alfa | Procrit ® |
| Aldesleukin | Proleukin, IL-2 ® |
| Somatrem | Protropin ® |
| dornase alfa | Pulmozyme ® |
| Efalizumab: selective, reversible T-cell blocker | RAPTIVA ™ |
| combination of ribavirin and alpha interferon | Rebetron ™ |
| Interferon beta 1a | Rebif ® |
| antihemophilic factor | Recombinate ® rAHF/ |
| antihemophilic factor | ReFacto ® |
| Lepirudin | Refludan ® |
| Infliximab | REMICADE ® |
| Abciximab | ReoPro ™ |
| Reteplase | Retavase™ |
| Rituxima | Rituxan ™ |
| interferon alfa-2^{a} | Roferon-A ® |
| Somatropin | Saizen ® |
| synthetic porcine secretin | SecreFlo ™ |
| Basiliximab | Simulect ® |
| Eculizumab | SOLIRIS (R) |
| Pegvisomant | SOMAVERT ® |
| Palivizumab; recombinantly produced, humanized mAb | Synagis ™ |
| thyrotropin alfa | Thyrogen ® |
| Tenecteplase | TNKase ™ |
| Natalizumab | TYSABRI ® |
| human immune globulin intravenous 5% and 10% solutions | Venoglobulin-S ® |
| interferon alfa-n1, lymphoblastoid | Wellferon ® |
| drotrecogin alfa | Xigris ™ |
| Omalizumab; recombinant DNA-derived humanized monoclonal | Xolair ® |
| antibody targeting immunoglobulin-E | |
| Daclizumab | Zenapax ® |
| ibritumomab tiuxetan | Zevalin ™ |
| Somatotropin | Zorbitive ™ (Serostim ®) |

In embodiments, the polypeptide is a hormone, blood clotting/coagulation factor, cytokine/growth factor, antibody molecule, fusion protein, protein vaccine, or peptide as shown in Table 2.

TABLE 2
Exemplary Products

| Therapeutic | | |
|---|---|---|
| Product type | Product | Trade Name |
| Hormone | Erythropoietin, Epoein-α | Epogen, Procrit |
| | Darbepoetin-α | Aranesp |
| | Growth hormone (GH), | Genotropin, Humatrope, Norditropin, |
| | somatotropin | NovIVitropin, Nutropin, Omnitrope, |
| | | Protropin, Siazen, Serostim, Valtropin |
| | Human follicle-stimulating | Gonal-F, Follistim |
| | hormone (FSH) | |
| | Human chorionic | Ovidrel |
| | gonadotropin | |
| | Lutropin-α | Luveris |
| | Glucagon | GlcaGen |
| | Growth hormone releasing | Geref |
| | hormone (GHRH) | |
| | Secretin | ChiRhoStim (human peptide), SecreFlo |
| | | (porcine peptide) |
| | Thyroid stimulating | Thyrogen |
| | hormone (TSH), thyrotropin | |
| Blood | Factor VIIa | NovoSeven |
| Clotting/Coagulation | Factor VIII | Bioclate, Helixate, Kogenate, |
| Factors | | Recombinate, ReFacto |
| | Factor IX | Benefix |
| | Antithrombin III (AT-III) | Thrombate III |
| | Protein C concentrate | Ceprotin |
| Cytokine/Growth | Type I alpha-interferon | Infergen |
| factor | Interferon-αn3 (IFNαn3) | Alferon N |
| | Interferon-ß1a (rIFN-ß) | Avonex, Rebif |
| | Interferon-ß1b (rIFN-ß) | Betaseron |
| | Interferon-γ1b (IFN γ) | Actimmune |
| | Aldesleukin (interleukin | Proleukin |
| | 2(IL2), epidermal | |
| | theymocyte activating | |
| | factor; ETAF | |
| | Palifermin (keratinocyte | Kepivance |
| | growth factor; KGF) | |
| | Becaplemin (platelet- | Regranex |
| | derived growth factor; | |
| | PDGF) | |
| | Anakinra (recombinant IL1 | Anril, Kineret |
| | antagonist) | |
| Antibody molecules | Bevacizumab (VEGFA | Avastin |
| | mAb) | |
| | Cetuximab (EGFR mAb) | Erbitux |
| | Panitumumab (EGFR mAb) | Vectibix |
| | Alemtuzumab (CD52 mAb) | Campath |
| | Rituximab (CD20 chimeric | Rituxan |
| | Ab) | |
| | Trastuzumab (HER2/Neu | Herceptin |
| | mAb) | |
| | Abatacept (CTLA Ab/Fc | Orencia |
| | fusion) | |
| | Adalimumab (TNF& mAb) | Humira |
| | Etanercept (TNF | Enbrel |
| | receptor/Fc fusion) | |
| | Infliximab (TNFα chimeric | Remicade |
| | mAb) | |
| | Alefacept (CD2 fusion | Amevive |
| | protein) | |
| | Efalizumab (CD11a mAb) | Raptiva |
| | Natalizumab (integrin α4 | Tysabri |
| | subunit mAb) | |
| | Eculizumab (C5mAb) | Soliris |
| | Muromonab-CD3 | Orthoclone, OKT3 |
| Other: | Insulin | Humulin, Novolin |
| Fusion | Hepatitis B surface antigen | Engerix, Recombivax HB |
| proteins/Protein | (HBsAg) | |
| vaccines/Peptides | HPV vaccine | Gardasil |
| | OspA | LYMErix |
| | Anti-Rhesus(Rh) | Rhophylac |
| | immunoglobulin G | |
| | Enfuvirtide | Fuzeon |
| | Spider silk, e.g., fibrion | QMONOS |

In embodiments, the protein is a multispecific protein, e.g., a bispecific antibody as shown in Table 3.

TABLE 3
Bispecific Formats

| Name (other | | | | | |
|---|---|---|---|---|---|
| names, | | | Proposed | | Diseases (or |
| sponsoring | BsAb | | mechanisms of | Development | healthy |
| organizations) | format | Targets | action | stages | volunteers) |
| Catumaxomab | BsIgG: | CD3, | Retargeting of T | Approved in | Malignant ascites |
| (Removab ® | Triomab | EpCAM | cells to tumor, Fc | EU | in EpCAM |
| Fresenius Biotech, | | | mediated effector | | positive tumors |
| Trion Pharma, | | | functions | | |
| Neopharm) | | | | | |
| Ertumaxomab | BsIgG: | CD3, HER2 | Retargeting of T | Phase I/II | Advanced solid |
| (Neovii Biotech, | Triomab | | cells to tumor | | tumors |
| Fresenius Biotech) | | | | | |
| Blinatumomab | BiTE | CD3, CD19 | Retargeting of T | Approved in | Precursor B-cell |
| (Blincyto ®, AMG | | | cells to tumor | USA | ALL |
| 103, MT 103, | | | | Phase II and | ALL |
| MEDI 538, | | | | III | DLBCL |
| Amgen) | | | | Phase II | NHL |
| | | | | Phase I | |
| REGN1979 | BsAb | CD3, CD20 | | | |
| (Regeneron) | | | | | |
| Solitomab (AMG | BiTE | CD3, | Retargeting of T | Phase I | Solid tumors |
| 110, MT110, | | EpCAM | cells to tumor | | |
| Amgen) | | | | | |
| MEDI 565 (AMG | BiTE | CD3, CEA | Retargeting of T | Phase I | Gastrointestinal |
| 211, MedImmune, | | | cells to tumor | | adenocancinoma |
| Amgen) | | | | | |
| RO6958688 | BsAb | CD3, CEA | | | |
| (Roche) | | | | | |
| BAY2010112 | BiTE | CD3, PSMA | Retargeting of T | Phase I | Prostate cancer |
| (AMG 212, Bayer; | | | cells to tumor | | |
| Amgen) | | | | | |
| MGD006 | DART | CD3, CD123 | Retargeting of T | Phase I | AML |
| (Macrogenics) | | | cells to tumor | | |
| MGD007 | DART | CD3, gpA33 | Retargeting of T | Phase I | Colorectal cancer |
| (Macrogenics) | | | cells to tumor | | |
| MGD011 | DART | CD19, CD3 | | | |
| (Macrogenics) | | | | | |
| SCORPION | BsAb | CD3, CD19 | Retargeting of T | | |
| (Emergent | | | cells to tumor | | |
| Biosolutions, | | | | | |
| Trubion) | | | | | |
| AFM11 (Affimed | TandAb | CD3, CD19 | Retargeting of T | Phase I | NHL and ALL |
| Therapeutics) | | | cells to tumor | | |
| AFM12 (Affimed | TandAb | CD19, CD16 | Retargeting of NK | | |
| Therapeutics) | | | cells to tumor | | |
| | | | cells | | |
| AFM13 (Affimed | TandAb | CD30, | Retargeting of NK | Phase II | Hodgkin's |
| Therapeutics) | | CD16A | cells to tumor | | Lymphoma |
| | | | cells | | |
| GD2 (Barbara Ann | T cells | CD3, GD2 | Retargeting of T | Phase I/II | Neuroblastoma |
| Karmanos Cancer | preloaded with BsAb | | cells to tumor | | and |
| Institute) | | | | | osteosarcoma |
| pGD2 (Barbara | T cells | CD3, Her2 | Retargeting of T | Phase II | Metastatic breast |
| Ann Karmanos | preloaded | | cells to tumor | | cancer |
| Cancer Institute) | with BsAb | | | | |
| EGFRBi-armed | T cells | CD3, EGFR | Autologous | Phase I | Lung and other |
| autologous | preloaded | | activated T cells | | solid tumors |
| activated T cells | with BsAb | | to EGFR-positive | | |
| (Roger Williams | | | tumor | | |
| Medical Center) | | | | | |
| Anti-EGFR-armed | T cells | CD3, EGFR | Autologous | Phase I | Colon and |
| activated T-cells | preloaded | | activated T cells | | pancreatic |
| (Barbara Ann | with BsAb | | to EGFR-positive | | cancers |
| Karmanos Cancer | | | tumor | | |
| Institute) | | | | | |
| rM28 (University | Tandem | CD28, | Retargeting of T | Phase II | Metastatic |
| Hospital Tübingen) | scFv | MAPG | cells to tumor | | melanoma |
| IMCgp100 | ImmTAC | CD3, peptide | Retargeting of T | Phase I/II | Metastatic |
| (Immunocore) | | MHC | cells to tumor | | melanoma |
| DT2219ARL | 2 scFv | CD19, CD22 | Targeting of | Phase I | B cell leukemia |
| (NCI, University of | linked to | | protein toxin to | | or lymphoma |
| Minnesota) | diphtheria | | tumor | | |
| | toxin | | | | |
| XmAb5871 | BsAb | CD19, | | | |
| (Xencor) | | CD32b | | | |
| NI-1701 | BsAb | CD47, CD19 | | | |
| (NovImmune) | | | | | |
| MM-111 | BsAb | ErbB2, | | | |
| (Merrimack) | | ErbB3 | | | |
| MM-141 | BsAb | IGF-1R, | | | |
| (Merrimack) | | ErbB3 | | | |
| NA (Merus) | BsAb | HER2, | | | |
| | | HER3 | | | |
| NA (Merus) | BsAb | CD3, | | | |
| | | CLEC12A | | | |
| NA (Merus) | BsAb | EGFR, | | | |
| | | HER3 | | | |
| NA (Merus) | BsAb | PD1, | | | |
| | | undisclosed | | | |
| NA (Merus) | BsAb | CD3, | | | |
| | | undisclosed | | | |
| Duligotuzumab | DAF | EGFR, | Blockade of 2 | Phase I and II | Head and neck |
| (MEHD7945A, | | HER3 | receptors, ADCC | Phase II | cancer |
| Genentech, Roche) | | | | | Colorectal cancer |
| LY3164530 (Eli | Not | EGFR, MET | Blockade of 2 | Phase I | Advanced or |
| Lily) | disclosed | | receptors | | metastatic cancer |
| MM-111 | HSA body | HER2, | Blockade of 2 | Phase II | Gastric and |
| (Merrimack | | HER3 | receptors | Phase I | esophageal |
| Pharmaceuticals) | | | | | cancers |
| | | | | | Breast cancer |
| MM-141, | IgG-scFv | IGF-1R, | Blockade of 2 | Phase I | Advanced solid |
| (Merrimack | | HER3 | receptors | | tumors |
| Pharmaceuticals) | | | | | |
| RG7221 | CrossMab | Ang2, VEGFA | Blockade of 2 | Phase I | Solid tumors |
| (RO5520985, | | | proangiogenics | | |
| Roche) | | | | | |
| RG7716 (Roche) | CrossMab | Ang2, VEGFA | Blockade of 2 | Phase I | Wet AMD |
| | | | proangiogenics | | |
| OMP-305B83 | BsAb | DLL4/VEGF | | | |
| (OncoMed) | | | | | |
| TF2 | Dock and | CEA, HSG | Pretargeting | Phase II | Colorectal, |
| (Immunomedics) | lock | | tumor for PET or | | breast and lung |
| | | | radioimaging | | cancers |
| ABT-981 | DVD-Ig | IL-1α, IL-1ß | Blockade of 2 | Phase II | Osteoarthritis |
| (AbbVie) | | | proinflammatory | | |
| | | | cytokines | | |
| ABT-122 | DVD-Ig | TNF, IL-17A | proinflammatory | Phase II | Rheumatoid |
| (Abb Vie) | | | Blockade of 2 | | arthritis |
| | | | cytokines | | |
| COVA322 | IgG- | TNF, IL17A | Blockade of 2 | Phase I/II | Plaque psoriasis |
| | fynomer | | proinflammatory | | |
| | | | cytokines | | |
| SAR156597 | Tetravalent | IL-13, IL-4 | Blockade of 2 | Phase I | Idiopathic |
| (Sanofi) | bispecific | | proinflammatory | | pulmonary |
| | tandem IgG | | cytokines | | fibrosis |
| GSK2434735 | Dual- | IL-13, IL-4 | Blockade of 2 | Phase I | (Healthy |
| (GSK) | targeting | | proinflammatory | | volunteers) |
| | domain | | cytokines | | |
| Ozoralizumab | Nanobody | TNF, HSA | Blockade of | Phase II | Rheumatoid |
| (ATN103, Ablynx) | | | proinflammatory | | arthritis |
| | | | cytokine, binds to | | |
| | | | HSA to increase | | |
| | | | half-life | | |
| ALX-0761 (Merck | Nanobody | IL-17A/F, | Blockade of 2 | Phase I | (Healthy |
| Serono, Ablynx) | | HSA | proinflammatory | | volunteers) |
| | | | cytokines, binds | | |
| | | | to HSA to | | |
| | | | increase half-life | | |
| ALX-0061 | Nanobody | IL-6R, HSA | Blockade of | Phase I/II | Rheumatoid |
| (AbbVie, Ablynx; | | | proinflammatory | | arthritis |
| | | | cytokine, binds to | | |
| | | | HSA to increase | | |
| | | | half-life | | |
| ALX-0141 | Nanobody | RANKL, | Blockade of bone | Phase I | Postmenopausal |
| (Ablynx, | | HSA | resorption, binds | | bone loss |
| Eddingpharm) | | | to HSA to | | |
| | | | increase half-life | | |
| RG6013/ACE910 | ART-Ig | Factor IXa, | Plasma | Phase II | Hemophilia |
| (Chugai, Roche) | | factor X | coagulation | | |

TABLE 4

| Protein Product | Reference Listed Drug |
|---|---|
| interferon gamma-1b | Actimmune ® |
| alteplase; tissue plasminogen activator | Activase ®/Cathflo ® |
| Recombinant antihemophilic factor | Advate |
| human albumin | Albutein ® |
| Laronidase | Aldurazyme ® |
| Interferon alfa-N3, human leukocyte derived | Alferon N ® |
| human antihemophilic factor | Alphanate ® |
| virus-filtered human coagulation factor IX | AlphaNine ® SD |
| Alefacept; recombinant, dimeric fusion | Amevive ® |
| protein LFA3-Ig | |
| Bivalirudin | Angiomax ® |
| darbepoetin alfa | Aranesp ™ |
| Bevacizumab | Avastin ™ |
| interferon beta-1a; recombinant | Avonex ® |
| coagulation factor IX | BeneFix ™ |
| Interferon beta-1b | Betaseron ® |
| Tositumomab | BEXXAR ® |
| antihemophilic factor | Bioclate ™ |
| human growth hormone | BioTropin ™ |
| botulinum toxin type A | BOTOX ® |
| Alemtuzumab | Campath ® |
| acritumomab; technetium-99 labeled | CEA-Scan ® |
| alglucerase; modified form of beta- | Ceredase ® |
| glucocerebrosidase | |
| imiglucerase; recombinant form of beta- | Cerezyme ® |
| glucocerebrosidase | |
| crotalidae polyvalent immune Fab, ovine | CroFab ™ |
| digoxin immune fab [ovine] | DigiFab ™ |
| Rasburicase | Elitek ® |
| Etanercept | ENBREL ® |
| epoietin alfa | Epogen ® |
| Cetuximab | Erbitux ™ |
| algasidase beta | Fabrazyme ® |
| Urofollitropin | Fertinex ™ |
| follitropin beta | Follistim ™ |
| Teriparatide | FORTEO ® |
| human somatropin | GenoTropin ® |
| Glucagon | GlucaGen ® |
| follitropin alfa | Gonal-F ® |
| antihemophilic factor | Helixate ® |
| Antihemophilic Factor; Factor XIII | HEMOFIL |
| adefovir dipivoxil | Hepsera ™ |
| Trastuzumab | Herceptin ® |
| Insulin | Humalog ® |
| antihemophilic factor/von Willebrand factor | Humate-P ® |
| complex-human | |
| Somatotropin | Humatrope ® |
| Adalimumab | HUMIRA ™ |
| human insulin | Humulin ® |
| recombinant human hyaluronidase | Hylenex ™ |
| interferon alfacon-1 | Infergen ® |
| Eptifibatide | Integrilin ™ |
| alpha-interferon | Intron A ® |
| Palifermin | Kepivance |
| Anakinra | Kineret ™ |
| antihemophilic factor | Kogenate ® FS |
| insulin glargine | Lantus ® |
| granulocyte macrophage colony-stimulating | Leukine ®/Leukine ® |
| factor | Liquid |
| lutropin alfa for injection | Luveris |
| OspA lipoprotein | LYMErix ™ |
| Ranibizumab | LUCENTIS ® |
| gemtuzumab ozogamicin | Mylotarg ™ |
| Galsulfase | Naglazyme ™ |
| Nesiritide | Natrecor ® |
| Pegfilgrastim | Neulasta ™ |
| Oprelvekin | Neumega ® |
| Filgrastim | Neupogen ® |
| Fanolesomab | NeutroSpec ™ (formerly |
| | LeuTech ®) |
| somatropin [rDNA] | Norditropin ®/Norditropin |
| | Nordiflex ® |
| Mitoxantrone | Novantrone ® |
| insulin; zinc suspension; | Novolin L ® |
| insulin; isophane suspension | Novolin N ® |
| insulin, regular; | Novolin R ® |
| Insulin | Novolin ® |
| coagulation factor VIIa | NovoSeven ® |
| Somatropin | Nutropin ® |
| immunoglobulin intravenous | Octagam ® |
| PEG-L-asparaginase | Oncaspar ® |
| abatacept, fully human soluable fusion | Orencia ™ |
| protein | |
| muromomab-CD3 | Orthoclone OKT3 ® |
| high-molecular weight hyaluronan | Orthovisc ® |
| human chorionic gonadotropin | Ovidrel ® |
| live attenuated Bacillus Calmette-Guerin | Pacis ® |
| peginterferon alfa-2a | Pegasys ® |
| pegylated version of interferon alfa-2b | PEG-Intron ™ |
| Abarelix (injectable suspension); | Plenaxis ™ |
| gonadotropin-releasing hormone | |
| Antagonist | |
| epoietin alfa | Procrit ® |
| Aldesleukin | Proleukin, IL-2 ® |
| Somatrem | Protropin ® |
| dornase alfa | Pulmozyme ® |
| Efalizumab; selective, reversible T-cell | RAPTIVA ™ |
| blocker | |
| combination of ribavirin and alpha interferon | Rebetron ™ |
| Interferon beta 1a | Rebif ® |
| antihemophilic factor | Recombinate ® rAHF/ |
| antihemophilic factor | ReFacto ® |
| Lepirudin | Refludan ® |
| Infliximab | REMICADE ® |
| Abciximab | ReoPro ™ |
| Reteplase | Retavase ™ |
| Rituxima | Rituxan ™ |
| interferon alfa-2ª | Roferon-A ® |
| Somatropin | Saizen ® |
| synthetic porcine secretin | SecreFlo ™ |
| Basiliximab | Simulect ® |
| Eculizumab | SOLIRIS (R) |
| Pegvisomant | SOMAVERT ® |
| Palivizumab; recombinantly produced, | Synagis ™ |
| humanized mAb | |
| thyrotropin alfa | Thyrogen ® |
| Tenecteplase | TNKase ™ |
| Natalizumab | TYSABRI ® |
| human immune globulin intravenous 5% and | Venoglobulin-S ® |
| 10% solutions | |
| interferon alfa-n1, lymphoblastoid | Wellferon ® |
| drotrecogin alfa | Xigris ™ |
| Omalizumab; recombinant DNA-derived | Xolair ® |
| humanized monoclonal | |
| antibody targeting immunoglobulin-E | |
| Daclizumab | Zenapax ® |
| ibritumomab tiuxetan | Zevalin ™ |
| Somatotropin | Zorbtive ™ (Serostim ®) |

In some embodiments, the polypeptide is an antigen expressed by a cancer cell. In some embodiments the recombinant or therapeutic polypeptide is a tumor-associated antigen or a tumor-specific antigen. In some embodiments, the recombinant or therapeutic polypeptide is selected from HER2, CD20, 9-O-acetyl-GD3, βhCG, A33 antigen, CA19-9 marker, CA-125 marker, calreticulin, carboanhydrase IX (MN/CA IX), CCR5, CCR8, CD19, CD22, CD25, CD27, CD30, CD33, CD38, CD44v6, CD63, CD70, CC123, CD138, carcinoma embryonic antigen (CEA; CD66e), desmoglein 4, E-cadherin neoepitope, endosialin, ephrin A2 (EphA2), epidermal growth factor receptor (EGFR), epithelial cell adhesion molecule (EpCAM), ErbB2, fetal acetylcholine receptor, fibroblast activation antigen (FAP), fucosyl GM1, GD2, GD3, GM2, ganglioside GD3, Globo H, glycoprotein 100, HER2/neu, HER3, HER4, insulin-like growth factor receptor 1, Lewis-Y, LG, Ly-6, melanoma-specific chondroitin-sulfate proteoglycan (MCSCP), mesothelin, MUCl, MUC2, MUC3, MUC4, MUC5AC, MUC5_{B}, MUC7, MUC16, Mullerian inhibitory substance (MIS) receptor type II, plasma cell antigen, poly SA, PSCA, PSMA, sonic hedgehog (SHH), SAS, STEAP, sTn antigen, TNF-alpha precursor, and combinations thereof.

In some embodiments, the polypeptide is an activating receptor and is selected from 2B4 (CD244), α₄β₁ integrin, β₂ integrins, CD2, CD16, CD27, CD38, CD96, CD100, CD160, CD137, CEACAM1 (CD66), CRTAM, CS1 (CD319), DNAM-1 (CD226), GITR (TNFRSFi8), activating forms of KIR, NKG2C, NKG2D, NKG2E, one or more natural cytotoxicity receptors, NTB-A, PEN-5, and combinations thereof, optionally wherein the 3₂ integrins comprise CD11a-CD 18, CD11 b-CD 18, or CD11c-CD 18, optionally wherein the activating forms of KIR comprise KlR2DS1, KIR2DS4, or KIR-S, and optionally wherein the natural cytotoxicity receptors comprise NKp30, NKp44, NKp46, or NKp80.

In some embodiments, the polypeptide is an inhibitory receptor and is selected from KIR, ILT2/LIR-1/CD85j, inhibitory forms of KIR, KLRG1, LAIR-1, NKG2A, NKR-PiA, Siglec-3, Siglec-7, Siglec-9, and combinations thereof, optionally wherein the inhibitory forms of KIR comprise KIR2DL1, KIR2DL2, KIR2DL3, KIR3DL1, KIR3DL2, or KIR-L.

In some embodiments, the polypeptide is an activating receptor and is selected from CD3, CD2 (LFA2, OX34), CD5, CD27 (TNFRSF7), CD28, CD30 (TNFRSF8), CD40L, CD84 (SLAMF5), CD137 (4-1BB), CD226, CD229 (Ly9, SLAMF3), CD244 (2B4, SLAMF4), CD319 (CRACC, BLAME), CD352 (Lyl08, NTBA, SLAMF6), CRTAM (CD355), DR3 (TNFRSF25), GITR (CD357), HVEM (CD270), ICOS, LIGHT, LTOR (TNFRSF3), OX40 (CD134), NKG2D, SLAM (CD150, SLAMFi), TCRα, TCRβ, TCRδγ, TIM1 (HAVCR, KIM1), and combinations thereof.

In some embodiments, the polypeptide is an inhibitory receptor and is selected from PD-1 (CD279), 2B4 (CD244, SLAMF4), B71 (CD80), B7H1 (CD274, PD-L1), BTLA (CD272), CD160 (BY55, NK28), CD352 (Ly108, NTBA, SLAMF6), CD358 (DR6), CTLA-4 (CD152), LAG3, LAIR1, PD-1H (VISTA), TIGIT (VSIG9, VSTM3), TIM2 (TIMD2), TIM3 (HAVCR2, KIM3), and combinations thereof.

Other exemplary proteins include, but are not limited to any protein described in Tables 1-10 of Leader et al., “Protein therapeutics: a summary and pharmacological classification”, Nature Reviews Drug Discovery, 2008, 7:21-39 (incorporated herein by reference); or any conjugate, variant, analog, or functional fragment of the recombinant polypeptides described herein.

Other recombinant protein products include non-antibody scaffolds or alternative protein scaffolds, such as, but not limited to: DARPins, affibodies and adnectins. Such non-antibody scaffolds or alternative protein scaffolds can be engineered to recognize or bind to one or two, or more, e.g., 1, 2, 3, 4, or 5 or more, different targets or antigens.

### Variable Diameter Bioreactors

The methods of preparation of products, e.g., product variants, disclosed herein can be used with variable diameter bioreactor vessels (VDBs). Variable diameter bioreactor vessels are described herein and can include a first vessel section having a first diameter configured to hold a liquid media and biologic material and a second vessel section having a second diameter that is greater than the first diameter such that the liquid media can be increased from a first volume to a second volume within the vessel. In some aspects, the first vessel section can have an aspect ratio of greater than 0.3:1. In some aspects, the second vessel section can have an aspect ratio of greater than 0.3:1. In some aspects, the liquid media comprises an inoculant. The first vessel section can be configured to be an initial inoculation stage bioreactor. The second vessel section can be configured to be a growth stage or seed bioreactor. The variable diameter bioreactor vessel can further include at least one agitator. In some aspects the bioreactor can further include at least one of an agitator shaft, an impeller, a sparger, a probe port, a fill port, a condenser, a vent filter, a foam breaker plate, a sample port, a level probe, and a load cell. In some aspects, the variable diameter bioreactor vessel can be configured for growing mammalian, insect, plant or microbial cells.

In other aspects, a variable diameter bioreactor system for use with the methods of preparation of products, e.g., product variants, disclosed herein can include a bioreactor vessel having a first diameter and a second diameter such that the diameter of the vessel varies along a height of the vessel, an agitator disposed within the bioreactor vessel such that the agitator provides desired agitation at a given liquid height of the bioreactor vessel, and a control system operable to scale up the bioreactor vessel from a first volume to a second volume. In some aspects, the first vessel section has an aspect ratio of greater than 0.3:1 and the second vessel section has an aspect ratio of greater than 0.3:1. The first section of the vessel can be an initial inoculation stage bioreactor. The second section of the vessel can be a growth stage vessel section. The variable diameter bioreactor system can also include a sparger, a probe port, a fill port, a condenser, a vent filter, a foam breaker plate, a sample port, a level probe, and/or a load cell. In some aspects, the variable diameter bioreactor system is configured for mammalian cell production.

In other aspects, a method of preparation of products, e.g., product variants includes inoculating a bioreactor at a first volume with a growth media and inoculum and adding additional growth media to the bioreactor to scale up the bioreactor volume to a second volume following completion of an inoculation stage. In some aspects, the method can further include adding additional growth media to the bioreactor to scale up the bioreactor volume to a third volume following completion of a growth stage. In some aspects, the inoculum is a mammalian cell. In other aspects, the bioreactor can have a minimum aspect ratio of 0.3:1.

Bioreactor processing of biologic material-such as microbial and mammalian cultures in Variable Diameter Bioreactors (VDB), such as those described herein, is designed to sustain growth conditions starting with a minimal inoculum, utilize a continuous and/or bolus, medium and/or feed addition over the growth duration to sustain cell growth, and obtain a sufficient volume of culture for producing the desired product. By accomplishing cell growth and production in a single VDB, multiple smaller volume bioreactors can be eliminated. A single VDB will reduce the overall footprint of bioreactor equipment needed for production of desired product, eliminate multiple seed reactors, multiple CIP's, SIP's, start up operations, post run operations and minimizes non-logarithmic cell growth or lag phase effect currently observed with the use of multiple seed bioreactors thus simplifying the overall facility operation resulting in time and cost savings.

For example a single 20,000 L VDB can replace a 200 L N-3, 1000 L N-2 and 5000 L N-1 seed bioreactor. It is also estimated that the replacement of 3 seed bioreactors by a single VDB can save greater than 300 square foot of clean room space.

In some aspects, utilizing a conical or smaller diameter cylindrical geometry for the lower portion of the bioreactor and a cylindrical design for the upper portion allows for controllable scale-up within one bioreactor providing key design benefits in relation to mixing and aeration. For example, using a variable diameter conical or smaller diameter cylindrical bottomed tank, with an aspect ratio of greater than 1:1 (liquid height to vessel width at liquid level) can be maintained to support minimal inoculation volume with sufficient liquid head for oxygen transfer during bulk up to larger volume culture. The culture volume can then be bulked up through addition of media to sustain cell growth. The alternative bottom design can enable a higher aspect ratio and ability to operate at lower volumes compared to typical fixed diameter cylindrical tank bioreactor designs.

As used herein, “biologic material” is understood to mean particles consisting, in all or in part, of cellular or viral material, either living or dead, and/or products produced and expressed by cellular or viral cultures. For example, this can include eukaryotic or prokaryotic cells, such as bacteria, mammalian, plant, fungal, viruses such as talimogene laherparepvec (T-VEC), or any other desired therapeutic or biochemical product. In some aspects, “biologic material” includes cells produced for cellular therapy programs. In some aspects, “biologic material” includes viruses produced for virotherapy including viral gene therapy, viral immunotherapy, or protozoal virotherapy. In some aspects, “biologic material” includes cellular or viral cultures for fermentation production of products, e.g., product variants, as described herein. In some aspects, the biologic material can include inert material such as a substrate or immobilization material. Moreover, as used herein, “liquid media” is understood to mean any liquid typically used in bioreactor processes such as growth media, water, inoculum, and biologic material. The liquid media can have solid particles and/or gas suspended, emulsified, entrained, or otherwise present in the liquid media.

As is shown in the Figures, variable diameter bioreactors can have multiple configurations that allow for the efficient scale-up from inoculum to seed and production within a single bioreactor vessel. In some aspects, variable diameter bioreactors can have more suitable aspect ratios when bioreactor media volume is low relative to traditional vertical cylinder uniform diameter reactors. The addition of media or feed from low volume inoculation up to production volume also provides a stabilized environment for cell growth as waste is diluted and fresh nutrients are continuously introduced and mixed. In some aspects, example variable diameter bioreactors can be configured for fermentation processes and can be batch, fed-batch, or continuous or perfusion production, and the method of production can change depending upon the stage of culture and volume stage within the bioreactor vessel. For example, during the initial inoculation stage, a batch or fed-batch process can be used. Then, once the cell-growth stage has reached maturity and the bioreactor volume is scaled up to its desired limit, a continuous or perfusion process could be utilized. The variable diameter bioreactors described herein can be formed of any suitable material and can be configured for single-use, disposable systems. In some aspects, the reactors can be configured for use in mono-type systems or in multiproduct suites.

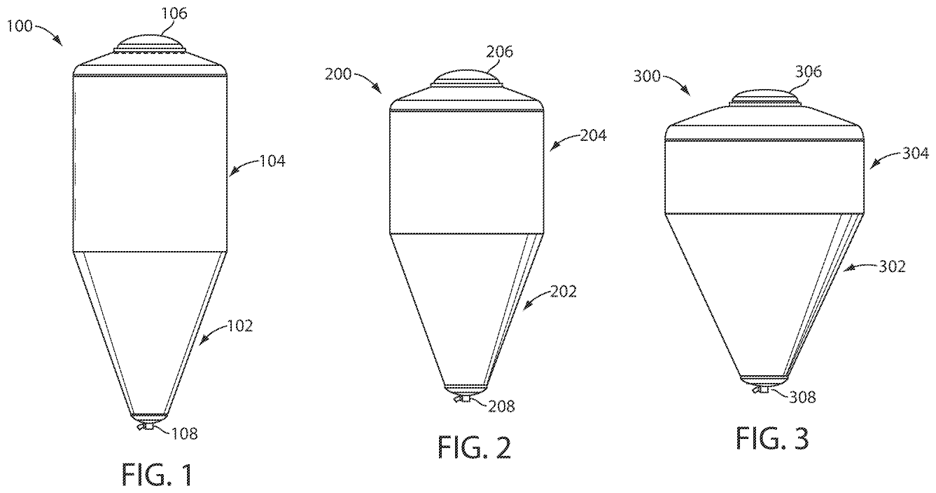

Further, Variable Diameter Bioreactors can be configured to have any desired total volume. As will be discussed in more detail, VDB's can have about 20,000 liters (L) total volume but it is also possible to design a VDB with 1,000 L total volume, for example, or even 10 L total volume. For example, a 10 L total volume VDB could also be used for process development or scale down studies whereas a 1000 L volume can serve as a pilot scale bioreactor. FIGS. 1-3 illustrate example variable diameter bioreactors having a conical lower portion and a cylindrical upper portions whereby the height of the upper cylindrical portions are varied to achieve various desired volumes.

FIG. 1 illustrates a variable diameter bioreactor (VDB) 100. The variable diameter bioreactor 100 comprises a first vessel section 102 having a first diameter configured to hold a liquid media or culture of biologic material such as appropriate cells and a second vessel section 104 having a second diameter that is greater than the first diameter such that the liquid media can be increased from a first volume to a second volume within the vessel 100. The variable diameter bioreactor 100 also has at least one inlet, such as a manway 106, and at least one outlet 108.

FIG. 2 illustrates a variable diameter bioreactor (VDB) 200 with a decreased height of an upper cylindrical portion relative to the height of the upper cylindrical portion of the variable diameter bioreactor shown in FIG. 1. The variable diameter bioreactor 200 comprises a first vessel section 202 having a first diameter configured to hold a liquid media and a second vessel section 204 having a second diameter that is greater than the first diameter. The variable diameter bioreactor 200 also has at least one inlet, such as a manway 206, and at least one outlet 208.

FIG. 3 illustrates a variable diameter bioreactor (VDB) 300 with a decreased height of an upper cylindrical portion relative to the height of the upper cylindrical portion of the variable diameter bioreactor shown in FIG. 2. The variable diameter bioreactor 300 comprises a first vessel section 302 having a first diameter configured to hold a liquid media and a second vessel section 304 having a second diameter that is greater than the first diameter. The variable diameter bioreactor 300 also has at least one inlet, such as a manway 306, and at least one outlet 308.

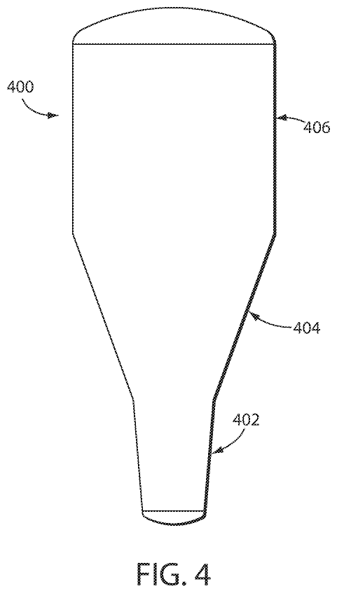

FIG. 4 illustrates a variable diameter bioreactor (VDB) 400. The variable diameter bioreactor 400 comprises a first vessel section 402, a second vessel section 404, and a third vessel section 406. The first vessel section has a diameter that varies along the height of the vessel-that is, the diameter of the first vessel section 402 and the diameter of the second vessel section 404 increases towards the top of the bioreactor 400. As shown, however, the diameter of the third section 406 stays relatively uniform throughout the section 406.

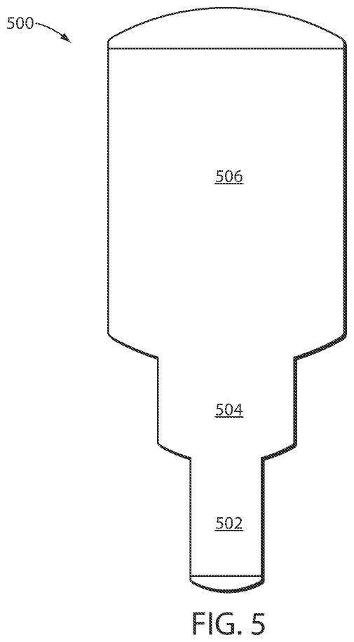

FIG. 5 illustrates a variable diameter bioreactor (VDB) 500. The variable diameter bioreactor 500 comprises a first vessel section 502, a second vessel section 504, and a third vessel section 506. The first vessel section has a diameter that varies along the height of the vessel in a step-wise fashion-that is with movement up the vessel the diameter of the third vessel section 506 is greater than the volume of the second vessel section 504, which is greater than the volume of the first vessel section 502. As is shown, in this aspect, the diameter of each stage is uniform throughout the stage with a step increase between the first stage 502 and second stage 504, and another step increase in diameter between second stage 504 and third stage 506.

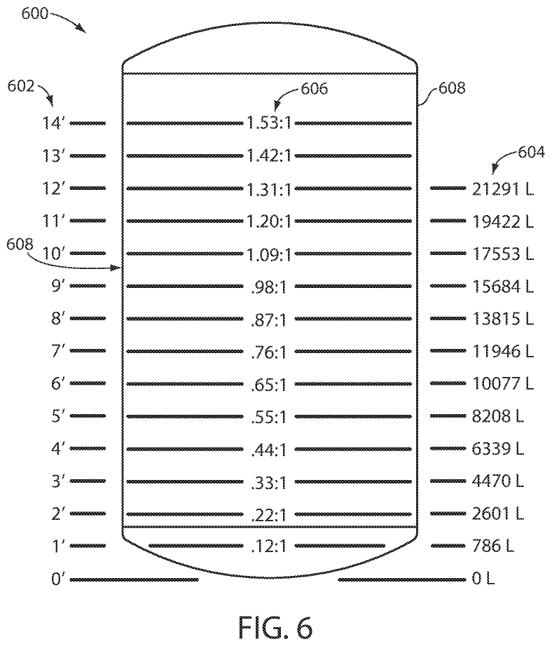

FIGS. 6-9 illustrate example aspect ratios and volumes of various bioreactor designs. As described above, aspect ratio is defined as vessel height to width or diameter. As shown, the reactors of FIGS. 6-9 can have volumes ranging between about 0 liters and 25,000 liters (L).

FIG. 6 is a typical bioreactor 600 having a uniform diameter (i.e., not a variable diameter bioreactor). The typical bioreactor 600 has only a single vessel section 608 and has a bioreactor height 602, volume 604, and aspect ratio 606. The typical bioreactor 600 has the bioreactor height 602, and aspect ratio 606 shown in Table 1. As shown, at low volumes, e.g. 800 L, the aspect ratio of typical uniform diameter reactors is significantly lower than 0.3. Further, uniform diameter bioreactors need to be operated at an aspect ratio of at least 0.65 or higher, which in FIG. 6 represents a volume of about 10,000 L. Thus a uniform diameter bioreactor requires multiple seed bioreactors of progressively increasing culture volumes so as to achieve the desired culture volume for optimal operation

TABLE 1
Typical Bioreactor 600

| Height in Feet | Aspect Ratio |
|---|---|
| (602) | (606) |
| 0 | 0 |
| 1 | 0.12:1 |
| 2 | 0.22:1 |
| 3 | 0.33:1 |
| 4 | 0.44:1 |
| 5 | 0.55:1 |
| 6 | 0.65:1 |
| 7 | 0.76:1 |
| 8 | 0.87:1 |
| 9 | 0.98:1 |
| 10 | 1.09:1 |
| 11 | 1.20:1 |
| 12 | 1.31:1 |
| 13 | 1.42:1 |
| 14 | 1.53:1 |

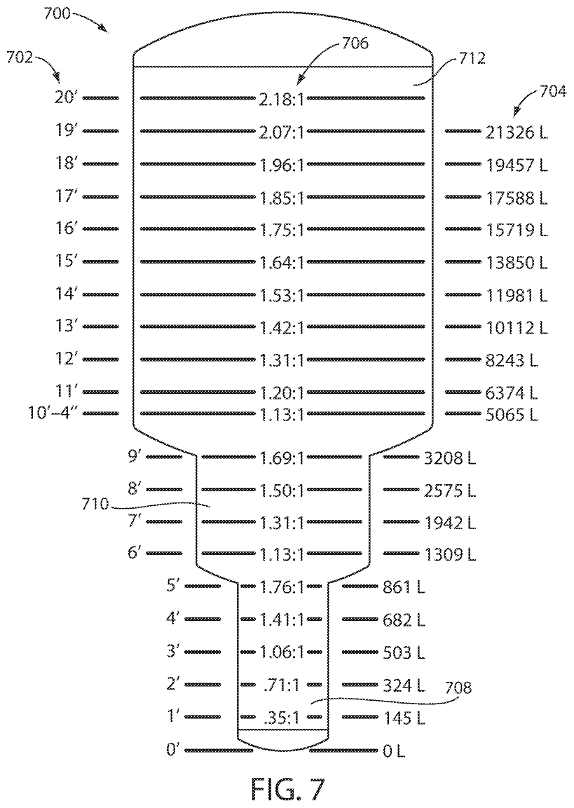

FIGS. 7, 8 and 9 show variable diameter bioreactors of different configurations all capable of operating at the desired volumes required to eliminate multiple seed bioreactors of 200 L, 1000 L and 4000 L respectively.

FIG. 7 illustrates an example variable diameter bioreactor (VDB) 700 having a bioreactor height 702, volume 704, and aspect ratio 706. As shown, the bioreactor 700 has a first vessel section 708, a second vessel section 710, and a third vessel section 712. Example bioreactor 700 has the bioreactor height 702, aspect ratio 706, and volume 704 shown in Table 2.

TABLE 2
VDB Bioreactor 700

| Height in Feet | Aspect Ratio |
|---|---|
| (702) | (706) |
| 0 | 0 |
| 1 | 0.35:1 |
| 2 | 0.71:1 |
| 3 | 1.06:1 |
| 4 | 1.41:1 |
| 5 | 1.76:1 |
| 6 | 1.13:1 |
| 7 | 1.31:1 |
| 8 | 1.50:1 |
| 9 | 1.69:1 |
| 10 | 1.13:1 |
| 11 | 1.20:1 |
| 12 | 1.31:1 |
| 13 | 1.42:1 |
| 14 | 1.53:1 |
| 15 | 1.64:1 |
| 16 | 1.75:1 |
| 17 | 1.85:1 |
| 18 | 1.96:1 |
| 19 | 2.07:1 |
| 20 | 2.18:1 |

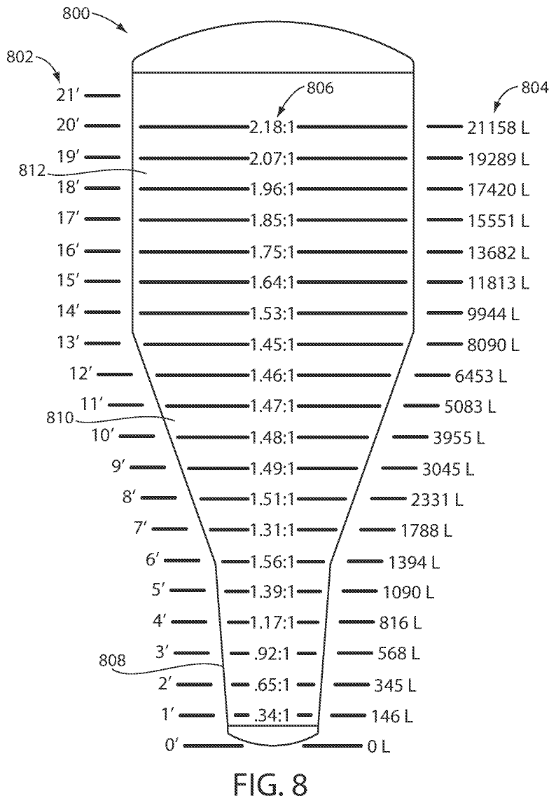

FIG. 8 illustrates an example variable diameter bioreactor (VDB) 800 having a bioreactor height 802, volume 804, and aspect ratio 806. As shown, the bioreactor 800 has a first vessel section 808, a second vessel section 810, and a third vessel section 812.

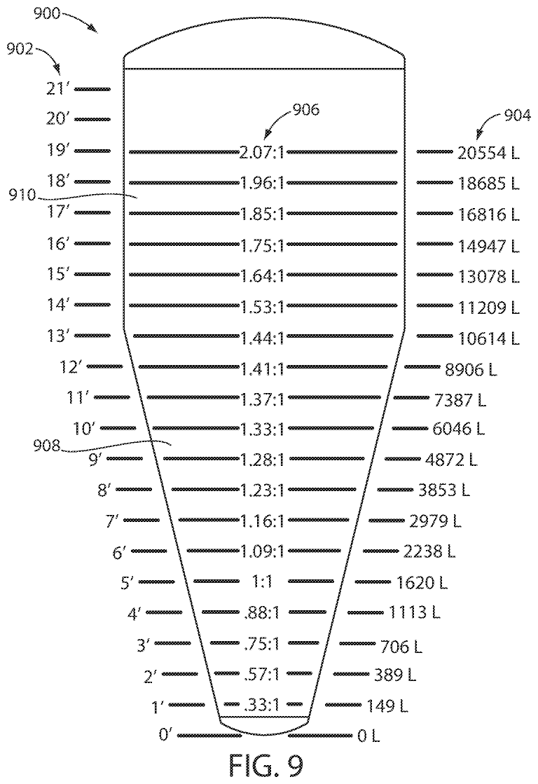

FIG. 9 illustrates an example variable diameter bioreactor (VDB) 900 having a bioreactor height 902, volume 904, and aspect ratio 906. As shown, the bioreactor 900 has a first vessel section 908, and a second vessel section 910. Example reactors 800, 900 have the bioreactor height 802, 902 and aspect ratio 806, 906 shown in Table 3.

TABLE 3
VDB Bioreactor 800 & 900

| Height in | Bioreactor 800 | Bioreactor 900 |
|---|---|---|
| Feet (802, | Aspect | Aspect |
| 902) | Ratio (806) | Ratio (906) |
| 0 | | |
| 1 | 0.34:1 | 0.33:1 |
| 2 | 0.65:1 | 0.57:1 |
| 3 | 0.92:1 | 0.75:1 |
| 4 | 1.17:1 | 0.88:1 |
| 5 | 1.39:1 | 1:1 |
| 6 | 1.56:1 | 1.09:1 |
| 7 | 1.31:1 | 1.16:1 |
| 8 | 1.51:1 | 1.23:1 |
| 9 | 1.49:1 | 1.28:1 |
| 10 | 1.48:1 | 1.33:1 |
| 11 | 1.47:1 | 1.37:1 |
| 12 | 1.46:1 | 1.41:1 |
| 13 | 1.45:1 | 1.44:1 |
| 14 | 1.53:1 | 1.53:1 |
| 15 | 1.64:1 | 1.64:1 |
| 16 | 1.75:1 | 1.75:1 |
| 17 | 1.85:1 | 1.85:1 |
| 18 | 1.96:1 | 1.96:1 |
| 19 | 2.07:1 | 2.07:1 |

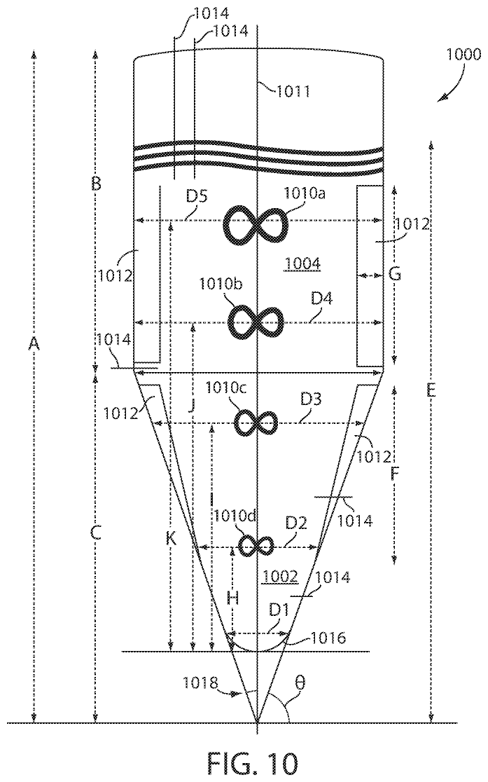

FIGS. 10 and 11 illustrate example variable diameter bioreactor vessel 1000 and 1100. As is shown, the variable diameter bioreactors 1000, 1200 can have a variety of ports, probes, spargers and other components such as at least one of an agitator shaft, an impeller, a sparger, a probe port, a fill port, a condenser, a vent filter, a foam breaker plate, a sample port, a level probe, and a load cell.

FIG. 10 is a schematic of VDB 1000 having a first vessel section 1002 and a second vessel section 1004. In some aspects, the first vessel section 1002 has a diameter that increases such that the first vessel section 1002 is a cone shape. The second vessel section 1004 can have a constant diameter such that it has a cylindrical shape. As shown, the VDB 1000 can have a total bioreactor height A. In some aspects, the total bioreactor height A can be in the range of about 5 feet to about 50 feet. For example, total bioreactor height can be about 20 feet. Additionally, as shown, an upper portion of the bioreactor can have a height B, the lower portion can have a height C, and the bioreactor can have a liquid height E. The liquid height E can vary based upon what stage of production is desired. In some aspects, the diameter of the lower portion can vary along height C and in some aspects the diameter of the upper portion can remain constant along height B.

As described herein, the diameter of the VDB bioreactor can vary as with movement along the total bioreactor height A or lower portion height C. As shown, the first vessel section 1002 can have a diameter that increases as a function of the lower portion height C. Movement up the reactor height A increases the diameter,for example to a second diameter D2, third diameter D3, and fourth diameter D4. In some non-limiting aspects, for example, D1 can be about 1 feet to about 3 feet, D2 can be about 1 feet to about 5 feet, D3 can be about 2 feet to about 10 feet, and D4 can be about 3 feet to about 20 feet. By way of one non-limiting example, the VDB bioreactor height A can be about 20 feet with a lower portion height C (cone height) of about 15 ft, an upper portion diameter (D4) of about 10 ft, a bottom diameter (D1) of about 2 ft, a D2 of about 3.25 feet, and a D3 of about 4.8 feet, yielding about a 24,909 liter (L) total volume, 13,789 L lower portion (cone) volume, and 11,120 L upper portion (cylinder) volume. Note that in some aspects, such as is shown in FIG. 10, the upper portion can have a uniform diameter such that D4 is equal to D5. Moreover, as shown the lower portion can have a cone shape having an angle θ that can be any angle suitable to provide the desired diameters and volumes for the lower portion. It is appreciated that the volume capacity can have a dished bottom 1016 and it is appreciated that the angled vertex 1018 is shown merely for explanatory purposes and need not be present in the reactor.

Moreover, the VDB 1000 includes a plurality of agitator impellers 1010a, 1010b, 1010c, and 1010d. The agitator impellers can be configured to provide agitation configured for the particular vessel section 1002, 1004 that the particular agitator impeller 1010a, 1010b, 1010c, and 1010d is disposed in. As shown, impeller 1010d can be disposed within the bioreactor at a height H, impeller 1010c can be disposed within the bioreactor at a height I, impeller 1010b can be disposed within the bioreactor at a height J, and impeller 1010a can be at a height K. For example heights H, I, J, K can be in the range of about 1 foot to about 20 feet. In some aspects, the agitators can have a single drive (not shown) that is disposed along the midpoint 1011 of the VDB 1000. In some aspects, the VDB 1000 can include baffles 1012 throughout the bioreactor 1000. As shown, the baffles 1012 can extend along a height G or F of the bioreactor. In some aspects, the VDB 1000 can include a plurality of ports 1014. The ports 1014 can be configured to be inlets, outlets, probes such as pH, temperature, oxygen, or any other desired probe or sensor. VDB 1000 can also include a single impeller.

FIG. 11 is a schematic of an example VDB bioreactor 1100. The VDB bioreactor 1100 has an inlet port 1102 and a bottom outlet valve 1104 configured to add and remove bioreactor media. The VDB bioreactor 1100 can have a first vessel section 1102, a second vessel section 1104, and a third vessel section 1106. The bioreactor has an agitator 1108 that includes a lower agitator 1110, a middle agitator 1112, an upper agitator 1114, and an agitator motor and drive 1116. Moreover, the bioreactor can include at least one sparger 1118 configured to allow for air or other nutrients to be bubbled through the bioreactor liquid media. Additionally, the bioreactor can include at least one probe or addition port 1120. The bioreactor can also include at least one CIP port 1122. As shown, the bioreactor can be configured to have a sparger 1118, probe and addition port 1120, and CIP port 1122 in each of the vessel sections 1102, 1104, 1106. The bioreactor can include any suitable control system for controlling the bioreactor systems including monitoring and controlling sparging, liquid media addition and removal, cell growth and production, oxygen levels, volumes, temperature, pH, and any other desired component. In some aspects, the control system is configured to scale up the bioreactor volume in either a continuous or batchwise manner. Additionally, the bioreactor can have at least one baffle 1124 disposed therein that is configured to provide suitable mixing conditions without causing undue stress on the bioreactor inoculum, which can lead to apoptosis. Additionally, the bioreactor can include a heat transfer shell 1126 which can have external insulation. VDB 1100 can also include a single impeller.

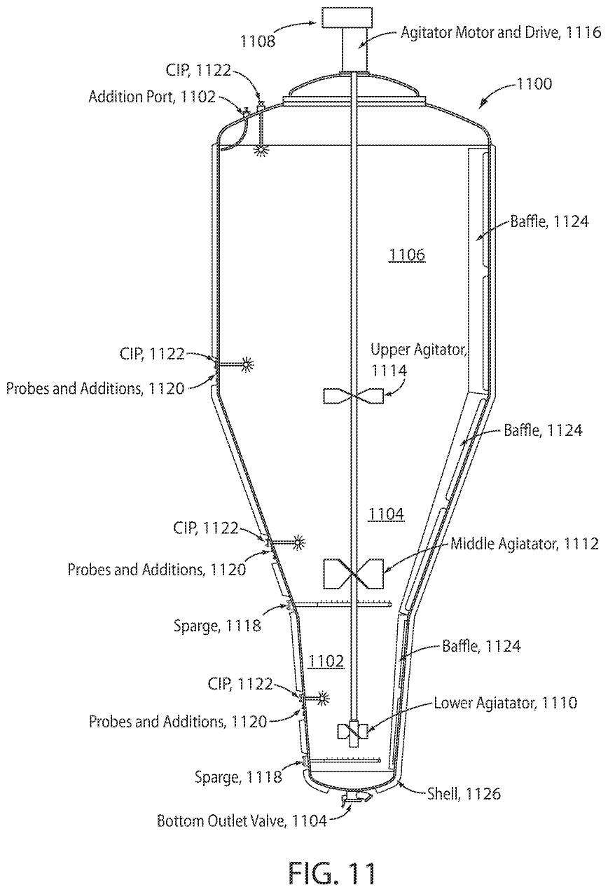

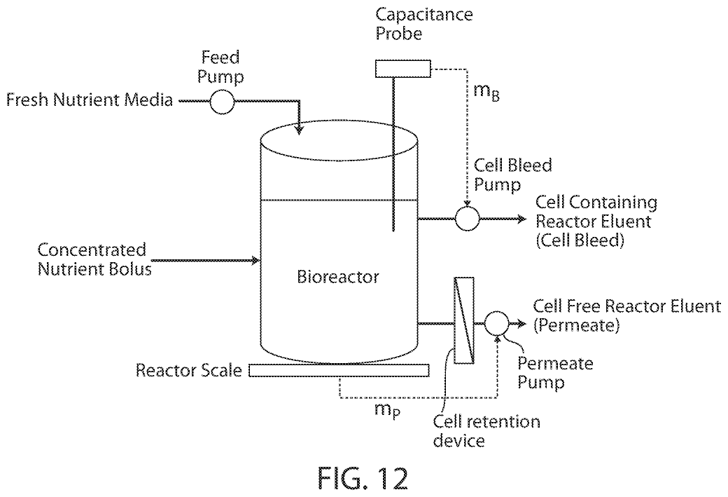

FIG. 12 is a schematic of an exemplary perfusion bioreactor, e.g., for obtaining a steady state and/or pseudo-steady state culture (e.g., in which cell density, nutrients, waste byproducts, and/or product charge variants are held constant over time). In some embodiments, the perfusion bioreactor can be designed as a modified Continuous Stirred Tank Reactor (CSTR), in which fresh nutrient media is fed into a main culture tank at a constant rate, e.g., through a feed pump. In some embodiments, a concentrated nutrient bolus can be added to the tank, e.g., to quickly or instantly alter the concentration of one or more nutrients, e.g., to a desired concentration. The volume of the tank's contents can be held at constant, for example, by constantly removing material from the tank, e.g., at a rate commensurate with that of fresh nutrient media being added. A reactor scale may, in some embodiments, be used to determine the quantity of material in the reactor tank. In some embodiments, a cell bleed pump is used to remove cell-containing reactor eluent (cell bleed) from the tank. In some embodiments, a permeate pump (e.g., behind a cell retention device) is used to remove cell-free reactor eluent (permeate) from the tank. A capacitance probe can be used, e.g., to monitor viable cell density, e.g., wherein capacitance measured at 1000 kHz is linearly correlated to an offline measurement of viable cell density.

In use, the variable diameter bioreactors described herein can be used to produce one or more products, e.g., one or more product variants, e.g., two, three, four, five, six, seven, eight, nine, ten, or more product variants, allowing for the efficient use of floor space by limiting the necessary reactors within a train to a single bioreactor. Specifically, the production of product, e.g., a product variant-can be achieved in a single VDB bioreactor by inoculating a bioreactor at a first volume with a growth media and inoculum and adding additional growth media to the bioreactor to scale up the bioreactor volume to a second volume following completion of an inoculation stage. In some aspects, use of the bioreactor can include adding additional growth media to the bioreactor to scale up the bioreactor volume to a third volume following completion of a growth stage.

That is, by condensing an inoculation bioreactor and all necessary follow-on growth or seed reactors into a single bioreactor vessel, the footprint of a particular plant is minimized. For example, for a 20,000 liter (L) desired production volume a single 20,000 L bioreactor can be used that consists of a first vessel section (i.e., inoculation vessel section), a second seed or growth section, and a third seed or growth vessel section. For example, the first vessel section (inoculation vessel section) can have a first diameter corresponding to about 100 L to about 200 L volume and a desired aspect ratio of between about 0.3:1 to about 2:1. Next, the second and third seed vessel sections can scale up the bioreactor volume to the desired 20,000 L quantity maintaining a range of desired aspect ratios. For example, the aspect ratios can remain between about 0.3:1 and about 3:1. The 20,000 L bioreactor unit can perform one or more, or all, of the following: feeding of nutrients and/or carbon sources, injection of suitable gas (e.g., oxygen), flow of fermentation or cell culture medium, separation of gas and liquid phases, maintenance of growth temperature, maintenance of pH level, agitation (e.g., stirring), and/or cleaning/sterilizing.

For example, this 20,000 L example bioreactor can be, in some aspects, inoculated at a first volume with a growth media and inoculum, such as a mammalian cell. In this inoculation stage, the reactor can be inoculated at a first volume such that the volume of the reactor is suitable for initial growth of the inoculum. Following a suitable period of time to allow the desired cell growth, the bioreactor can be scaled up to a second reactor volume to achieve a second growth stage of the inoculum. That is, additional growth media and any other desired component required for growth can be added to the bioreactor to scale up the bioreactor volume to a second volume following completion of the inoculation stage. This second volume can be any desired volume that is suitable for the desired continuing growth conditions needed for the inoculum. At this second volume further cell growth and proliferation can be achieved. In some aspects, a third, fourth, or any number of increasing volume growth stages can be utilized to continue the scaling up of the reactor volume to a desired volume.

In some aspects, a method of making a plurality of variant preparations, the plurality comprising at least a preparation of a first variant of a product (product variant 1) and a preparation of a second variant of a product (product variant 2), comprises:
(ai) generating a population of cells by inoculation of a first volume with culture, e.g., growth, medium and inoculum in a VDB and subsequent scaling up to a desired volume;
(aii) culturing the population of cells in culture medium under a first condition to form conditioned culture medium containing product variant 1;
(b) recovering product variant 1, e.g., a batch of product variant 1, (e.g., produced under the first condition);
(c) culturing the population of cells in culture medium under a second condition to form conditioned culture medium containing product variant 2;
(d) recovering product variant 2, e.g., a batch of product variant 2, (e.g., produced under the second condition);
   thereby providing a plurality of variant preparations, the plurality comprising at least a preparation of a first variant of a product (product variant 1) and a preparation of a second variant of a product (product variant 2),
   wherein product variant 1 (or a preparation of product variant 1) differs from product variant 2 (or a preparation of product variant 2) by a physical, chemical, biological, or pharmaceutical property.

### Numbered Embodiments

1. A method of making a plurality of variant preparations, the plurality comprising at least a preparation of a first variant of a product (product variant 1) and a preparation of a second variant of a product (product variant 2), comprising:
providing a population of cells in a vessel configured to allow cell culture;
(a-i) culturing the population of cells in culture medium under a first condition to form conditioned culture medium containing product variant 1;
(a-ii) recovering product variant 1 from culture;
(a-iii) optionally adding replacement medium to the conditioned culture medium;
(a-iv) optionally further culturing the population of cells under the first condition to produce additional conditioned medium;
(a-v) optionally recovering additional product variant 1;
(a-vi) optionally combining product variant 1 from (a-ii) and (a-v);
(b-i) culturing a population of cells in culture medium under a second condition to form conditioned culture medium containing product variant 2;
(b-ii) recovering product variant 2 from culture;
(b-iii) optionally adding replacement medium to the conditioned culture medium,
(b-iv) optionally further culturing the population of cells under the second condition to produce additional conditioned medium.
(b-v) optionally recovering additional,product variant 2;
(b-vi) optionally combining product variant 2 from (b-ii) and (b-v);
obtaining product variant 1 from a batch of product variant 1;
obtaining product variant 2 from a batch of product variant 2;
   thereby providing a plurality of variant preparations, the plurality comprising at least a preparation of a first variant of a product (product variant 1) and a preparation of a second variant of a product (product variant 2),
   wherein variant 1 (or a preparation of variant 1) differs from variant 2 (or a preparation of variant 2) by a physical, chemical, biological, or pharmaceutical property.

2. The method of paragraph 1, wherein the plurality comprises: a preparation of a third variant; a preparation of a fourth variant; a preparation of a fifth variant; a preparation of a sixth variant; a preparation of a seventh variant; a preparation of a eighth variant; a preparation of a ninth variant; a preparation of a tenth variant; a preparation of a eleventh variant; a preparation of a twelfth variant; a preparation of a thirteenth variant; a preparation of a fourteenth variant; a preparation of a fifteenth variant; a preparation of a sixteenth variant; a preparation of a seventeenth variant; a preparation of a eighteenth variant; a preparation of a nineteenth variant; and/or a preparation of a twentieth variant.

3. A method of making a plurality of variant preparations, the plurality comprising at least a preparation of a first variant of a product (product variant 1) and a preparation of a second variant of a product (product variant 2), comprising:
(a) culturing a population of cells in culture medium under a first condition to form conditioned culture medium containing product variant 1;
(b) recovering product variant 1;
(c) culturing the population of cells in culture medium under a second condition to form conditioned culture medium containing product variant 2;
(d) recovering product variant 2;
   thereby providing a plurality of variant preparations, the plurality comprising at least a preparation of a first variant of a product (product variant 1) and a preparation of a second variant of a product (product variant 2),
   wherein product variant 1 (or a preparation of product variant 1) differs from product variant 2 (or a preparation of product variant 2) by a physical, chemical, biological, or pharmaceutical property.

4. The method of paragraph 3, wherein the plurality comprises: a preparation of a third variant; a preparation of a fourth variant; a preparation of a fifth variant; a preparation of a sixth variant a preparation of a seventh variant; a preparation of a eighth variant; a preparation of a ninth variant; a preparation of a tenth variant; a preparation of a eleventh variant; a preparation of a twelfth variant; a preparation of a thirteenth variant; a preparation of a fourteenth variant; a preparation of a fifteenth variant; a preparation of a sixteenth variant; a preparation of a seventeenth variant; a preparation of a eighteenth variant; a preparation of a nineteenth variant; and/or a preparation of a twentieth variant.

5. The method of either of paragraphs 3 or 4, wherein recovering in step (b) comprises obtaining an aliquot of conditioned culture medium formed in step (a).

6. The method of paragraph 5, further comprising recovering product variant 1 from the aliquot of conditioned culture medium.

7. The method of paragraph 5, wherein step (b) further comprises adding replacement medium to the conditioned culture medium.

8. The method of paragraph 7, wherein the culture medium in (a) and the replacement medium are the same.

9. The method of paragraph 7, wherein the culture medium in (a) and the replacement medium differ from one another by one or more components such as chemical salts, metal and metal ions, amino acids, amino acid derivatives, sugars composition, hexosamines, n-acetylhexosamines, vitamins, lipids, polyamines, reducing/oxidizing agents, buffer composition, or hormones.

10. The method of any of paragraphs 7-9, wherein the volume of replacement culture medium is less than, equal to, or greater than the volume of the aliquot that is removed.

11. The method of any of paragraphs 7-10, wherein the population of cells in culture medium of (a) is comprised in a vessel.

12. The method of paragraph 11 wherein the volume of the aliquot removed, the replacement culture medium added, or both, are independently between 5 to 100% of the volume of the entire culture or of the capacity of the vessel.

13. The method of paragraph 11 wherein the amount removed, the replacement culture medium added, or both, are independently between 0.1 to 5 times the vessel volume per day of vessel operation.

14. The method of paragraph 7 wherein (b) further comprises further culturing the population of cells under the first condition to produce additional conditioned medium.

15. The method of either of paragraphs 7 or 14, comprising: (bii) recovering a second amount of product variant 1.

16. The method of paragraph 15, wherein recovering in step (bii) comprises obtaining an aliquot of further conditioned culture medium.

17. The method of either of paragraphs 15 or 16, comprising, adding replacement medium to the cultured medium of the previous step and repeating the steps of paragraphs 14 and 15, and optionally 16, e.g., repeating the steps of paragraphs 14 and 15, and optionally 16, X times, wherein X is 1, 2, 3, 4, 5, 6, 7, 8, 9, or 10.

18. The method of paragraph 17, wherein the culture medium in (a) and the replacement medium are the same.

19. The method of paragraph 17, wherein the culture medium in (a) and the replacement medium differ from one another by one or more components such as chemical salts, metal and metal ions, amino acids, amino acid derivatives, sugars composition, hexosamines, n-acetylhexosamines, vitamins, lipids, polyamines, reducing/oxidizing agents, buffer composition, or hormones.

20. The method of any of paragraphs 17-19, wherein the volume of replacement culture medium is less than, equal to, or greater than the volume of the aliquot that is removed.

21. The method of any of paragraphs 17-20 wherein the population of cells in culture medium of (a) is comprised in a vessel.

22. The method of paragraph 21, wherein the volume of the aliquot removed, the replacement culture medium added, or both, are independently between 5 to 100% of the volume of the entire culture or of the capacity of the vessel.

23. The method of paragraph 21, wherein the amount removed, the replacement culture medium added, or both, are independently between 0.1 to 5 times the vessel volume per day of vessel operation.

24. The method of any of paragraphs 15-23, comprising combining variant 1 obtained at different times.

25. The method of any of paragraphs 15-24, comprising combining a product variant 1 from a plurality of amounts, aliquots, or batches.

26. The method of any of paragraphs 1-25, wherein the population of cells is cultured under the first condition for 1 or more days.

27. The method of any of paragraphs 1-26, wherein, after a target value for a parameter is reached, the cell population is cultured under the second condition.

28. The method of paragraph 27, wherein the parameter is selected from: amount of product variant 1 produced, duration of culture under the first condition, or viability of culture.

29. The method of paragraph 28, wherein the parameter may further be selected from viable cell concentration.

30. The method of any of paragraphs 1-29, comprising manipulation of the medium or other condition to achieve the second condition.

31. The method of paragraph 30, wherein manipulation of the medium or other condition comprises altering one or more of: pH; level of dO₂; agitation; temperature; volume; density of the cell population; concentration of a component of the culture medium; agitation; the presence or amount of a nutrient, drug, inhibitor, or other chemical component (e.g., chemical salts, metal and metal ions, amino acids, amino acid derivatives, sugars composition, hexosamines, n-acetylhexosamines, vitamins, lipids, polyamines, reducing/oxidizing agents, buffer composition, or hormones).

32. The method of paragraph 31, comprising adding a different culture medium to the population of cells.

33. The method of any of paragraphs 1-32, wherein the culture of a population of cells is a perfusion production culture.

34. The method of paragraph 33, comprising interrupting perfusion as the medium transitions to a second condition.

35. The method of paragraph 33, comprising diverting perfusate to waste as the medium transitions to a second condition.

36. The method of any of paragraphs 1-35, wherein product variant 1 is removed from a downstream unit operation during production of product variant 2.

37. The method any of paragraphs 1-36, comprising culturing the cells until a target value for a parameter is reached.

38. The method of any of paragraphs 3-37, wherein recovering in step (d) comprises obtaining an aliquot of conditioned culture medium formed in step (c).

39. The method of paragraph 38, further comprising recovering product variant 2 from the aliquot of conditioned culture medium.

40. The method of paragraph 39, wherein step (d) further comprises adding replacement medium to the conditioned culture medium.

41. The method of paragraph 40, wherein the culture medium in (c) and the replacement medium are the same.

42. The method of paragraph 40, wherein the culture medium in (c) and the replacement medium differ from one another by one or more components such as chemical salts, metal and metal ions, amino acids, amino acid derivatives, sugars composition, hexosamines, n-acetylhexosamines, vitamins, lipids, polyamines, reducing/oxidizing agents, buffer composition, or hormones.

43. The method of any of paragraphs 40-42, wherein the volume of replacement culture medium is less than, equal to, or greater than the volume of the aliquot that is removed.

44. The method of any of paragraphs 40-43, wherein the population of cells in culture medium of (c) is comprised in a vessel.

45. The method of paragraph 44, wherein the volume of the aliquot removed, the replacement culture medium added, or both, are independently between 5 to 100% of the volume of the entire culture of the capacity of the vessel.

46. The method of paragraph 44 wherein the amount removed, the replacement culture medium added, or both, are independently between 0.1 to 5 times the vessel volume per day of vessel operation.

47. The method of paragraph 40, wherein (d) further comprises further culturing the population of cells under the second condition to produce additional conditioned medium.

48. The method of either of paragraphs 40 or 47, comprising (dii) recovering a second amount of product variant 2.

49. The method of paragraph 48, wherein recovering in step (dii) comprises obtaining an aliquot of further conditioned culture medium.

50. The method of either of paragraphs 48 or 49, comprising, adding replacement medium to the cultured medium of the previous step and repeating the steps of paragraphs 47 and 48, and optionally 49, e.g., repeating the steps of paragraphs 47 and 48, and optionally 49, X times, wherein X is 1, 2, 3, 4, 5, 6, 7, 8, 9, or 10.

51. The method of paragraph 50, wherein the culture medium in (c) and the replacement medium are the same.

52. The method of paragraph 50, wherein the culture medium in (c) and the replacement medium differ from one another by one or more components such as chemical salts, metal and metal ions, amino acids, amino acid derivatives, sugars composition, hexosamines, n-acetylhexosamines, vitamins, lipids, polyamines, reducing/oxidizing agents, buffer composition, or hormones.

53. The method of any of paragraphs 50-52, wherein the volume of replacement culture medium is less than, equal to, or greater than the volume of the aliquot that is removed.

54. The method of any of paragraphs 50-53, wherein the population of cells in culture medium of (c) is comprised in a vessel.

55. The method of paragraph 54, wherein the volume of the aliquot removed, the replacement culture medium added, or both, are independently between 5 to 100% of the volume of the entire culture or of the capacity of the vessel.

56. The method of any of paragraphs 50-53, wherein the amount removed, the replacement culture medium added, or both, are independently between 0.1 to 5 times the vessel volume per day of vessel operation.

57. The method of any of paragraphs 48-56, comprising combining variant 2 obtained at different times, e.g., a plurality of batches, aliquots, or amounts of product variant 2, e.g., combining the first and second amounts or batches of product variant 2.

58. The method of any of paragraphs 48-57, comprising combining product variant 2 from a plurality of amounts, aliquots, or batches.

59. The method of any paragraph 1-58, wherein the population of cells is cultured under the second condition for 1 or more days.

60. The method of any of paragraphs 1-59, wherein, after a target value for a parameter is reached, the cell population is cultured under a third condition.

61. The method of paragraph 60, wherein the parameter is selected from: amount of product variant 2 produced, duration of culture under the second condition, or viability of culture.

62. The method of paragraph 61, wherein the parameter may further be selected from viable cell concentration.

63. The method of any of paragraphs 60-62, comprising manipulation of the medium or other condition to achieve the third condition.

64. The method of paragraph 63, wherein manipulation of the medium or other condition comprises altering one or more of: pH; level of dO₂; agitation; temperature; volume; density of the cell population; concentration of a component of the culture medium; agitation; the presence or amount of a nutrient, drug, inhibitor or other chemical component (e.g., chemical salts, metal and metal ions, amino acids, amino acid derivatives, sugars composition, hexosamines, n-acetylhexosamines, vitamins, lipids, polyamines, reducing/oxidizing agents, buffer composition, or hormones).

65. The method of paragraph 64, comprising adding a different culture medium to the population of cells.

66. The method of any of paragraphs 1-65, wherein the culture of a population of cells is a perfusion production culture.

67. The method of paragraph 66, comprising interrupting perfusion as the medium transitions to a third condition.

68. The method of paragraph 66, comprising diverting perfusate to waste as the medium transitions to a third condition.

69. The method of any of paragraphs 1-68, wherein product variant 2 is removed from a downstream unit operation during production of product variant 3.

70. The method of any of paragraphs 1-69, comprising culturing the cells until a target value for a parameter is reached.

71. The method of any of paragraphs 1-70, wherein the plurality comprises a preparation of a third variant made under a third condition.

72. The method of paragraph 71, wherein the plurality comprises a preparation of a fourth variant made under a fourth condition, e.g., a preparation of a fourth variant made under a fourth condition made by the steps described herein for making the preparation of the first or second variant.

73. The method of paragraph 72, wherein the plurality comprises a preparation of a fifth variant made under a fifth condition, e.g., a preparation of a fifth variant made under a fifth condition made by the steps described herein for making the preparation of the first or second variant.

74. The method of paragraph 73, wherein the plurality comprises a preparation of an N^{th} variant made under a N^{th} condition, e.g., a preparation of a N^{th} variant made under a N^{th} condition made by the steps described herein for making the preparation of the first or second variant, wherein N is equal to or greater than 6, 7, 8, 9, 10, 11, 12, 13, 14, 15, 16, 17, 18, 19, 20, 21, 22, 23, 24, 25, 26, 27, 28, 29, or 30.

75. The method of either of paragraphs 1 or 2, wherein step (a) and step (b) are conducted in the same vessel, e.g., a production culture vessel.

76. The method of any of paragraphs 3-75, wherein steps (a) through (d) are conducted in the same vessel, e.g., a production culture vessel.

77. The method of paragraph 76, wherein the vessel is configured to allow operation in perfusion mode.

78. The method of paragraph 77, wherein the vessel is configured to allow removal of medium and addition of medium during culture.

79. The method of either of paragraphs 75 or 78, wherein the vessel comprises a variable diameter bioreactor.

80. The method of any of paragraphs 1-79, comprising purifying product variant 1.

81. The method of any of paragraphs 1-80, comprising purifying product variant 2.

82. The method of either of paragraphs 80 or 81, wherein a product variant is purified in a unit operation downstream from the vessel in which the population of cells is cultured.

83. The method of any of paragraphs 1-82, further comprising evaluating how a first product variant (or a preparation of the first product variant) differs from a second product variant (or a preparation of a second product variant), for one or more of:
glycosylation (e.g., galactosylation);
sialylation;
charge (e.g., pI);
sequence, e.g., N terminal or C terminal sequence,
homogeneity;
purity;
activity;
amount of inactive variant;
propensity to aggregate, or aggregation;
clarity;
deamidation;
glycation;
proline amidation;
disulfide heterogeneity;
dimerization;
protease susceptibility or proteolytic degradation; and
methionine oxidation.

84. The method of any of paragraphs 1-83, further comprising providing a preparation of product variant 1.

85. The method of any of paragraphs 1-84, further comprising providing a preparation of product variant 2.

86. The method of any of paragraphs 1-85, further comprising providing a plurality of preparations of different product variants.

87. The method of any of paragraphs 1-86, wherein a first product variant (or a preparation of the first variant) differs from a second product variant (or a preparation of a second product variant), by one or more of:
glycosylation (e.g., galactosylation);
sialylation;
charge (e.g., pI);
sequence, e.g., N terminal or C terminal sequence,
homogeneity;
purity;
activity;
amount of inactive variant;
propensity to aggregate, or aggregation;
clarity;
deamidation;
glycation;
proline amidation;
disulfide heterogeneity;
dimerization;
protease susceptibility or proteolytic degradation; and
methionine oxidation.

88. The method of any of paragraphs 1-87, comprising producing a plurality of product variants (or preparations of product variants), wherein 1, 2, 3, 4, 5, 6, 7. 8, 9, 10, 11, 12, 13, 14, 15, 16, 17, 18, 19, or 20 or more of the product variants (or preparations of product variant) each of which differ from one another by one or more of:
glycosylation (e.g., galactosylation);
sialylation;
charge (e.g., pI);
sequence, e.g., N terminal or C terminal sequence,
homogeneity;
purity;
activity;
amount of inactive variant;
propensity to aggregate, or aggregation;
clarity;
deamidation;
glycation;
proline amidation;
disulfide heterogeneity;
dimerization;
protease susceptibility or proteolytic degradation; and
methionine oxidation.

89. The method of paragraph 88, comprising producing 3 product variants (or preparations of product variant) each of which differs from one another by one or more of:
glycosylation (e.g., galactosylation);
sialylation;
charge (e.g., pI);
sequence, e.g., N terminal or C terminal sequence,
homogeneity;
purity;
activity;
amount of inactive variant;
propensity to aggregate, or aggregation;
clarity;
deamidation;
glycation;
proline amidation;
disulfide heterogeneity;
dimerization;
protease susceptibility or proteolytic degradation; and
methionine oxidation.

90. The method of any one of paragraphs 1-89, wherein the first and/or second conditions are steady-state conditions.

91. The method of any one of paragraphs 63-65, wherein the third condition is a steady-state condition.

92. The method of any one of paragraphs 30-32 or 63-65, wherein manipulation of the medium comprises adding a concentrated bolus of one or more of the following to the culture medium: a component of the culture medium, a nutrient, a drug, an inhibitor, or other chemical component, e.g., Lysine, Galactose, any water soluble copper compounds (e.g., Cuprous sulfate or copper chloride), any water soluble Manganese compounds (e.g., Manganese chloride), any water soluble Zinc compounds (e.g., Zinc chloride), any water soluble Iron compounds (e.g., Ferrous sulfate), N-acetyl mannosamine, Sodium Butyrate, N-acetylarginine, or L-arginine.

93. The method of any one of paragraphs 30-32 or 63-65, wherein manipulation of the medium comprises increasing the concentration of one or more of the following in the culture medium entering the reactor (e.g., replacement medium): a component of the culture medium, a nutrient, a drug, an inhibitor, or other chemical component, e.g., Lysine, Galactose, any water soluble copper compounds (e.g., Cuprous sulfate or copper chloride), any water soluble Manganese compounds (e.g., Manganese chloride), any water soluble Zinc compounds (e.g., Zinc chloride), any water soluble Iron compounds (e.g., Ferrous sulfate), N-acetyl mannosamine, Sodium Butyrate, N-acetylarginine, or L-arginine.

94. The method of any one of paragraphs 30-32 or 63-65, wherein manipulation of the medium comprises one or both of:
a) adding a concentrated bolus of a component to the culture medium, or
b) increasing the concentration of a component in the culture medium entering the reactor (e.g., replacement medium),
wherein the component is selected from one or more of: a component of the culture medium, a nutrient, a drug, an inhibitor, or other chemical component, e.g., Lysine, Galactose, any water soluble copper compounds (e.g., Cuprous sulfate or copper chloride), any water soluble Manganese compounds (e.g., Manganese chloride), any water soluble Zinc compounds (e.g., Zinc chloride), any water soluble Iron compounds (e.g., Ferrous sulfate), N-acetyl mannosamine, Sodium Butyrate, N-acetylarginine, or L-arginine.

95. The method of any of paragraphs 92-94 wherein manipulation of the medium comprises adding CuSO₄ to the culture medium (e.g., by adding a concentrated bolus of CuSO₄ to the culture medium, by increasing the concentration of CuSO₄ in the culture medium entering the reactor (e.g., replacement medium), or both).

96. The method of any of paragraphs 92-94, wherein manipulation of the medium comprises adding N-acetylarginine to the culture medium (e.g., by adding a concentrated bolus of N-acetylarginine to the culture medium, by increasing the concentration of N-acetylarginine in the culture medium entering the reactor (e.g., replacement medium), or both).

97. The method of any of paragraphs 92-94, wherein manipulation of the medium comprises adding lysine to the culture medium (e.g., by adding a concentrated bolus of lysine to the culture medium, by increasing the concentration of lysine in the culture medium entering the reactor (e.g., replacement medium), or both).

98. The method of any one of paragraphs 11-13, 21-23, 43-45, 53-55, or 73-77, wherein the vessel is a bioreactor, e.g., a perfusion bioreactor and/or variable diameter bioreactor.

99. A preparation of a variant product described herein or, made by, or makeable by, any of the methods of paragraphs 1-98.

100. A plurality of variant preparations, the plurality comprising at least a preparation of a first variant of a product (product variant 1) and a preparation of a second variant of a product (product variant 2), described herein, or made by, or makeable by, any of the methods of paragraphs 1-98.

101. A vessel, e.g., a bioreactor, e.g., a perfusion bioreactor and/or variable diameter bioreactor, charged with a mixture of cells described herein.

102. A method of evaluating the progress of a method for making a plurality of product variant preparations, comprising:
(a) culturing a population of cells in culture medium under a first condition to form conditioned culture medium containing a first product variant (product variant 1);
(b) acquiring a value for the progress of the method for making a plurality of product variant preparations toward one or more target parameters selected from: amount of product variant 1 produced, duration of culture under the first condition, or viability of culture;
(c) responsive to the value, determining the progress of the method for making a plurality of product variant preparations toward the one or more target parameters; and
(d) optionally, responsive to the determination that one or more target parameters has been reached, manipulating the culture medium or other condition to achieve a second condition,
thereby evaluating the progress of a method for making a plurality of product variant.

103. The method of paragraph 102, wherein the method for making a plurality of product variant preparations is the method of any one of paragraphs 1-98.

104. A method of modifying a method for producing a product variant, comprising:
(a) culturing a population of cells in culture medium under a first condition to form conditioned culture medium containing the product variant (product variant 1); amount of product variant 1 produced, duration of culture under the first condition, or viability of culture;
(c) responsive to the evaluation of the progress toward the one or more target parameters, manipulating the culture medium or other condition to achieve a second condition; and
(d) optionally, culturing the population of cells in culture medium under the second condition to form conditioned culture medium containing a second product variant (product variant 2),
thereby modifying the method for producing a product variant.

105. The method of any of paragraphs 102-104, wherein the target parameters of (b) further comprise viable cell concentration.

106. The method of any of paragraphs 102-105, wherein manipulation of the medium or other condition comprises altering one or more of: pH; level of dO₂; agitation; temperature; volume; density of the cell population; concentration of a component of the culture medium; agitation; the presence or amount of a nutrient, drug, inhibitor, or other chemical component, e.g., Lysine, Galactose, any water soluble copper compounds (e.g., Cuprous sulfate or copper chloride), any water soluble Manganese compounds (e.g., Manganese chloride), any water soluble Zinc compounds (e.g., Zinc chloride), any water soluble Iron compounds (e.g., Ferrous sulfate), N-acetyl mannosamine, Sodium Butyrate, N-acetylarginine, or L-arginine.

107. The method of paragraph 106, comprising adding a different culture medium to the population of cells.

108. The method of any one of paragraphs 102-107, wherein manipulation of the medium comprises adding a concentrated bolus of one or more of the following to the culture medium: a component of the culture medium, a nutrient, a drug, an inhibitor, or other chemical component, e.g., Lysine, Galactose, any water soluble copper compounds (e.g., Cuprous sulfate or copper chloride), any water soluble Manganese compounds (e.g., Manganese chloride), any water soluble Zinc compounds (e.g., Zinc chloride), any water soluble Iron compounds (e.g., Ferrous sulfate), N-acetyl mannosamine, Sodium Butyrate, N-acetylarginine, or L-arginine.

109. The method of any one of paragraphs 102-107, wherein manipulation of the medium comprises increasing the concentration of one or more of the following in the culture medium entering the reactor (e.g., replacement medium): a component of the culture medium, a nutrient, a drug, an inhibitor, or other chemical component, e.g., Lysine, Galactose, any water soluble copper compounds (e.g., Cuprous sulfate or copper chloride), any water soluble Manganese compounds (e.g., Manganese chloride), any water soluble Zinc compounds (e.g., Zinc chloride), any water soluble Iron compounds (e.g., Ferrous sulfate), N-acetyl mannosamine, Sodium Butyrate, N-acetylarginine, or L-arginine.

110. The method of any one of paragraphs 102-107, wherein manipulation of the medium comprises one or both of:
a) adding a concentrated bolus of a component to the culture medium, or
b) increasing the concentration of a component in the culture medium entering the reactor (e.g., replacement medium),
wherein the component is selected from one or more of: a component of the culture medium, a nutrient, a drug, an inhibitor, or other chemical component, e.g., Lysine, Galactose, any water soluble copper compounds (e.g., Cuprous sulfate or copper chloride), any water soluble Manganese compounds (e.g., Manganese chloride), any water soluble Zinc compounds (e.g., Zinc chloride), any water soluble Iron compounds (e.g., Ferrous sulfate), N-acetyl mannosamine, Sodium Butyrate, N-acetylarginine, or L-arginine.

111. The method of any one of paragraphs 1-98, wherein the recovered product variant 1 is at least 50, 60, 70, 80, 90, 95, 99, or 100% product variant 1 (e.g., by weight, volume, or molar ratio), e.g., as a percentage of total product recovered.

112. The method of any one of paragraphs 1-98, wherein the recovered product variant 2 is at least 50, 60, 70, 80, 90, 95, 99, or 100% product variant 2 (e.g., by weight, volume, or molar ratio), e.g., as a percentage of total product recovered.

113. The method of any one of paragraphs 1-98, wherein the recovered product variant 1 is enriched by at least 5, 10, 20, 30, 40, 50, 60, 70, 80, 90, or 100% for product variant 1 as compared to product produced from a population of cells and culture medium not cultured under the first condition.

114. The method of any one of paragraphs 1-98, wherein the recovered product variant 2 is enriched by at least 5, 10, 20, 30, 40, 50, 60, 70, 80, 90, or 100% for product variant 2 as compared to product produced from a population of cells and culture medium not cultured under the second condition.

115. The method of any one of paragraphs 1-98, or 111-114, further comprising, after recovery of product variant 1, evaluating the recovered product variant 1.

116. The method of paragraph 115, wherein evaluating the recovered product variant 1 comprises evaluating the level of one or more of product quality attributes selected from: glycosylation, sialylation, charge, sequence (e.g., N terminal or C terminal sequence), homogeneity, purity (e.g., recovered product is at least 50, 60, 70, 80, 90, 95, 99, or 100% product variant 1 (e.g., by weight, volume, or molar ratio, or as a percentage of total product recovered)), activity, amount of inactive variant, propensity to aggregate or for aggregation, clarity, deamidation, glycation, methionine oxidation, or amount of product variant 1 produced.

117. The method of any one of paragraphs 1-98, or 111-114, further comprising, after recovery of product variant 2, evaluating the recovered product variant 2.

118. The method of paragraph 117, wherein evaluating the recovered product variant 2 comprises evaluating the level of one or more of product quality attributes selected from: glycosylation, sialylation, charge, sequence (e.g., N terminal or C terminal sequence), homogeneity, purity (e.g., recovered product is at least 50, 60, 70, 80, 90, 95, 99, or 100% product variant 2 (e.g., by weight, volume, or molar ratio, or as a percentage of total product recovered)), activity, amount of inactive variant, propensity to aggregate or for aggregation, clarity, deamidation, glycation, methionine oxidation, or amount of product variant 2 produced.

119. The method of any of paragraphs 115-118, further comprising, responsive to the evaluation of the recovered product variant, determining whether to add replacement medium, further culture the population of cells under the current condition, or to culture the population of cells under a further condition.

120. The method of any of paragraphs 1-98, or 111-119, wherein the plurality of variant preparations is produced from a single production vessel, e.g., bioreactor, e.g., a perfusion bioreactor and/or variable diameter bioreactor.

121. The method of paragraph 120, wherein the plurality of variant preparations is produced continuously (e.g., maintaining conditions for active production without interrupting the culturing of the population of cells, e.g., to empty and/or clean the vessel) from a single production vessel, e.g., bioreactor, e.g., a perfusion bioreactor and/or variable diameter bioreactor.

122. The method of paragraph 121, wherein the plurality of variant preparations is produced in a shorter time than would have elapsed from making the plurality of variant preparations under similar conditions sequentially with interruptions (e.g., to empty, clean, and restart culturing of the population of cells under a further condition).

123. The method of paragraph 121, wherein the plurality of variant preparations is produced consuming or occupying fewer resources (e.g., equipment, culture, energy, or personnel) than would have been consumed or occupied from making the plurality of variant preparations under similar conditions sequentially with interruptions (e.g., to empty, clean, and restart culturing of the population of cells under a further condition).

124. The preparation of paragraph 99, formulated as a pharmaceutically effective composition.

125. A pharmaceutical composition comprising the preparation of paragraph 99.

126. The pharmaceutical composition of paragraph 125 comprising a pharmaceutically acceptable diluent, carrier, or excipient.

127. The plurality of variant preparations of paragraph 100, formulated as one or more pharmaceutically effective compositions.

128. A kit comprising the plurality of variant preparations of paragraph 100.

129. The kit of paragraph 128, wherein each variant preparation of the plurality is separately packaged into containers.

130. The kit of paragraph 129, wherein at least one container comprises product variant 1 that is at least 50, 60, 70, 80, 90, 95, 99, or 100% product variant 1 (e.g., by weight, volume, or molar ratio, or as a percentage of total product present).

131. The kit of paragraph 129, wherein at least one container comprises product variant 2 that is at least 50, 60, 70, 80, 90, 95, 99, or 100% product variant 2 (e.g., by weight, volume, or molar ratio, or as a percentage of total product present).

132. The kit of paragraph 128, wherein the kit comprises containers;
wherein at least one container comprises product variant 1 that is at least 50, 60, 70, 80, 90, 95, 99, or 100% product variant 1 (e.g., by weight, volume, or molar ratio, or as a percentage of total product present), or wherein at least one container comprises product variant 2 that is at least 50, 60, 70, 80, 90, 95, 99, or 100% product variant 2 (e.g., by weight, volume, or molar ratio, or as a percentage of total product present); and
wherein at least one container comprises a mixture of product variants 1 and 2.

133. The method of any of paragraphs 1-98, or 111-119, wherein the population of cells comprise at least one CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, CHO-GSKO cell, a CHOXceed cell, or a CHO-derived cell.

134. The method of any of paragraphs 1-98, or 111-119, wherein the population of cells comprise at least one HeIa, HEK293, HT1080, H9, HepG2, MCF7, Jurkat, NIH3T3, PC12, PER.C6, BHK (baby hamster kidney cell), VERO, SP2/0, NSO, YB2/0, YO, EB66, C127, L cell, COS (e.g., COS1 and COS7), QC1-3, CHOK1, CHOK1SV, Potelligent CHOK1SV, CHO GS knockout, CHOK1SV GS-KO, CHOS, CHO DG44, CHO DXB11, or CHOZN cell, or any cells derived therefrom.

135. The method of any of paragraphs 1-98, or 111-119, wherein the product variants are selected from one or more of the following: antibody molecules (e.g., monoclonal antibodies, bispecific antibodies), antibody mimetics (polypeptide molecules that bind specifically to antigens but that are not structurally related to antibodies such as e.g. DARPins, affibodies, adnectins, or IgNARs), fusion proteins (e.g., Fc fusion proteins, chimeric cytokines), other recombinant proteins (e.g., glycosylated proteins, enzymes, hormones), viral therapeutics (e.g., anti-cancer oncolytic viruses, viral vectors for gene therapy and viral immunotherapy), cell therapeutics (e.g., pluripotent stem cells, mesenchymal stem cells and adult stem cells), vaccines or lipid-encapsulated particles (e.g., exosomes, virus-like particles), RNA (such as e.g. siRNA) or DNA (such as e.g. plasmid DNA), antibiotics, or amino acids.

### EXEMPLIFICATION

### Example 1: Production of Homogenous Protein Products

The bioreactor unit, e.g., a variable diameter bioreactor, for use with the methods of the invention will be housed in an appropriate manufacturing environment, e.g. ISO Grade 7. The frozen cells will be thawed separately and connected to a first single use (SU) production vessel containing culture medium. The initiated culture will be grown under controlled conditions (e.g., temperature, pH, dissolved oxygen, agitation). Once sufficient culture is grown, e.g., the culture reaches a threshold density, it will be transferred to a production culture vessel containing culture medium. The production culture vessel will be capable of being operated in a perfusion mode. The initial conditions for the production culture (temperature, pH, dissolved oxygen, agitation, medium) will be capable of producing Product Variant 1. The perfusate containing Product Variant 1 will be purified through a series of downstream purification steps resulting in purified product 1. Purification or other post culture processes can be done concurrent with production of subsequent variants or after some or all of the some or all subsequent variants are produced.

Once sufficient quantity of product variant 1 is produced, the culture conditions will be manipulated, e.g., pH will be lowered or raised and/or a concentrated medium of different composition will be added to the bioreactor. Perfusion will either be interrupted and/or the perfusate will be diverted to waste. The downstream unit operations will be cleaned of product variant 1 via the use of appropriate cleaning and sanitizing buffers during this stage. Once the bioreactor has reached stable operation, collection and purification of perfusate can be commenced to produce product variant 2. It is envisioned that about 2 days will be required in order to produce each product variant, thus over a 30 day perfusion culture duration, 15 different product variants can be produced. The amount of each product variant produced can be adjusted either by culture duration or scale,e.g., perfusing higher volumes of. The invention is likely to employ a production culture in either a traditional stirred Bioreactor as shown in FIG. 12 or a Variable Diameter Bioreactor in order to provide for additional flexibility in operating the bioreactor at a wider range of culture volume.

With the methods described herein, one perfusion bioreactor can produce multiple product variants in a much shorter time period than a similar fed batch bioreactor or multiple fed batch reactors in parallel. At larger scale, e.g 2000 L, the duration from thawing frozen cells to initiation of production culture can take over 30 days with fed batch production. The methods described herein can reduce the time period to 2 days or less.

### Example 2: Production of Heterogeneous Protein Products in a Perfusion Reactor

A product, e.g., protein, can be produced in a single bioreactor such that multiple fractions or variants of the product, e.g., protein, are obtained, e.g., each with a different product quality attribute. In one example, these fractions are produced in a perfusion bioreactor apparatus.

An exemplary perfusion bioreactor apparatus, as shown in FIG. 12, may be designed to obtain a steady-state cell culture (which may be referred to as a pseudo-steady-state), in which at least one of cell density, nutrients, any chemical, waste byproducts, or product charge variants are held constant over time. The apparatus is designed as a modified Continuous Stirred Tank Reactor (CSTR), where fresh nutrient media is fed at a constant rate to the tank via a feed pump, and an amount of the tank contents is constantly removed such that the liquid level in the tank remains at a constant volume. The reactor volume is maintained at a constant value using a scale and a proportional-integral (PI) controller to control the total eluent rate (generally defined as the sum of the permeate and cell bleed pump rates). The PI controller specifies the total eluent rate needed to maintain the reactor mass set point.

Within the reactor, cells are grown in suspension culture. The cells are retained in the reactor apparatus via a cell retention device, located at the tank outlet flow. The cell retention device retains cells in the reactor, while allowing any liquid soluble components to freely exit the reactor via a permeate pump. Liquid soluble components include nutrients, waste byproducts, and protein product. Since the cells continuously divide under favorable conditions, a separate cell-containing reactor eluent stream is also employed, controlled by a cell bleed pump.

The reactor cell density was maintained at a constant set point by controlling the cell bleed pump rate. In this example, the viable cell density was measured using a capacitance probe, where capacitance measured at 1000 kHz was linearly correlated to an offline measurement of viable cell density. The viable cell density value, determined via the capacitance measurement, was fed into a PI controller, which determined a cell bleed pump rate. The cell bleed rate was determined by calculating a proportion of the total eluent flow, as prescribed by the reactor volume controller, to be the cell bleed. In this way, the cell density was controlled by removing cells via the cell bleed stream at a rate equal to the cell growth, as determined by a feed-back controller using capacitance to measure cell density.

The perfusion apparatus outlined above can be used to obtain multiple fractions of product with different product quality attributes. These fractions are produced by creating multiple steady-states, each producing product with different quality attribute(s). These discrete steady-state conditions are created by changing the concentration in the fresh nutrient feed of one or more nutrients or any other chemical known to influence the quality attribute(s) of the product. Once an initial steady-state is reached, (defined as constant cell density and product quality attribute levels), the reactor concentration of one or more nutrients or any other chemical which influences the product quality attribute is either raised or lowered, depending on the desired effect on the product quality attribute.

To raise the reactor concentration of a nutrient, a concentrated bolus of that nutrient is added to the reactor simultaneously while increasing the nutrient concentration in the fresh nutrient feed to the desired reactor concentration. The concentrated nutrient bolus contains enough nutrient to instantly raise the reactor concentration of the nutrient to the new desired steady-state concentration. In this way, a virtually instantaneous step increase in reactor nutrient concentration is obtained. Although the nutrient bolus is not necessary to achieve a steady-state product quality attribute fraction, it does serve to shorten the time required to do so. To lower the reactor concentration of a nutrient, the nutrient concentration in the fresh nutrient feed is simply lowered to the desired reactor concentration. Additional time is required in this case to obtain a new steady-state, as the reactor concentration of the nutrient must be allowed to reach a new steady-state value due to dilution dynamics.

### Charge Variants

In one example, the perfusion apparatus described above was used to obtain multiple fractions of product with different charge variant profiles, a product quality attribute for monoclonal antibodies (mAbs). To influence the charge variants profile of a product, any chemical or nutrient which has an effect on charge variants can be used. In this example, copper sulfate (CuSO₄) and lysine were used to influence the charge variants profile. The product was produced in this case using a GS−/− CHO cell line expressing a monoclonal antibody.

### Example 3: Shake Flask Experiments to Identify Chemical Compounds that Modulate Charge Variance

In one example, chemical compounds that can be used to modulate charge variance were identified. Three different chemicals (CuSO₄, lysine, and the carboxypeptidase inhibitor, N-acetylarginine) were evaluated. Briefly, Lonza GS^{-/-} CHO cells expressing a mAb were grown in shake flasks for 5 days in media supplemented with CuSO₄ (0.4, 1.2, 2.0 μM), lysine (10, 25, 50 mM), or N-acetylarginine (0.1, 1, 10 mM). On day 5, the media was harvested, the mAb was purified over Protein A resin, and the charge variance of the purified mAb was measured.

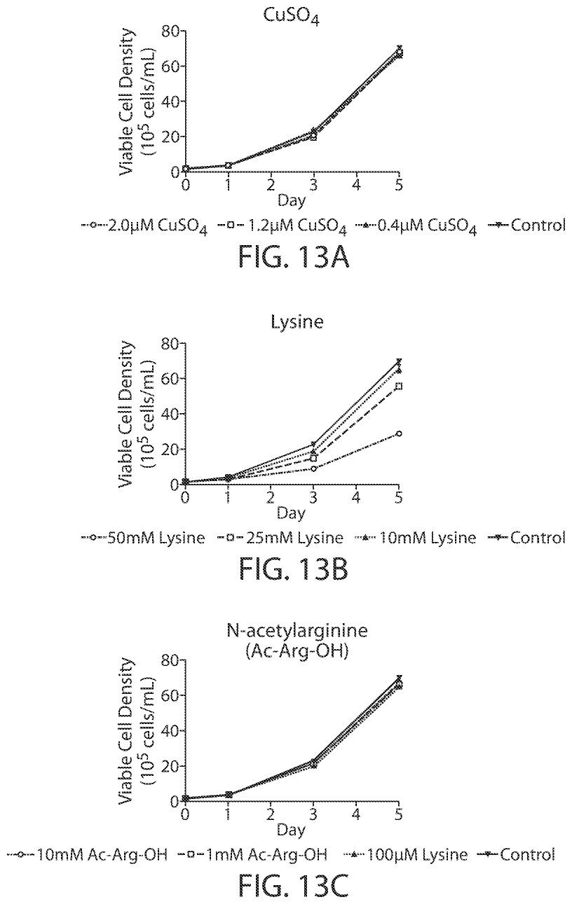

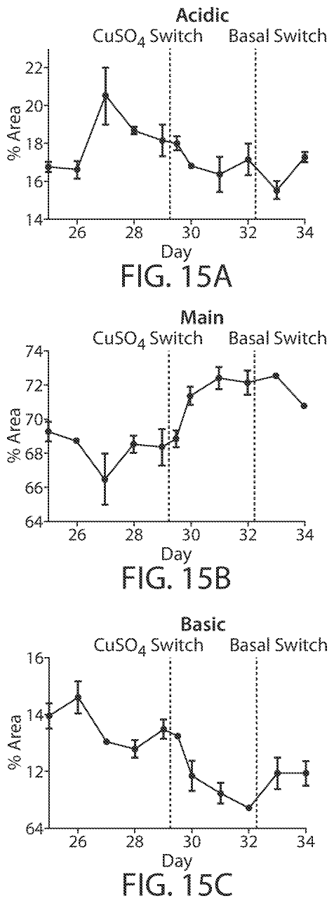

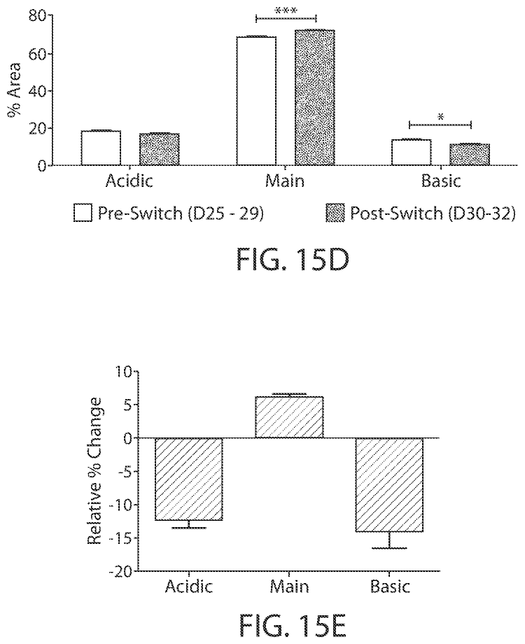

None of the tested doses of CuSO₄ affected either cell growth (FIG. 13A), or metabolic profile of the cultures. 2.0 μM CuSO₄ increased the abundance of the main peak (69±1.4%), while simultaneously decreasing the abundance of the basic peaks (17±1.3%), when compared to the absence of additional CuSO₄ (Main: 64±0.6%, p<0.01; Basic: 21±0.16%, p<0.01; FIGS. 15A-15C).

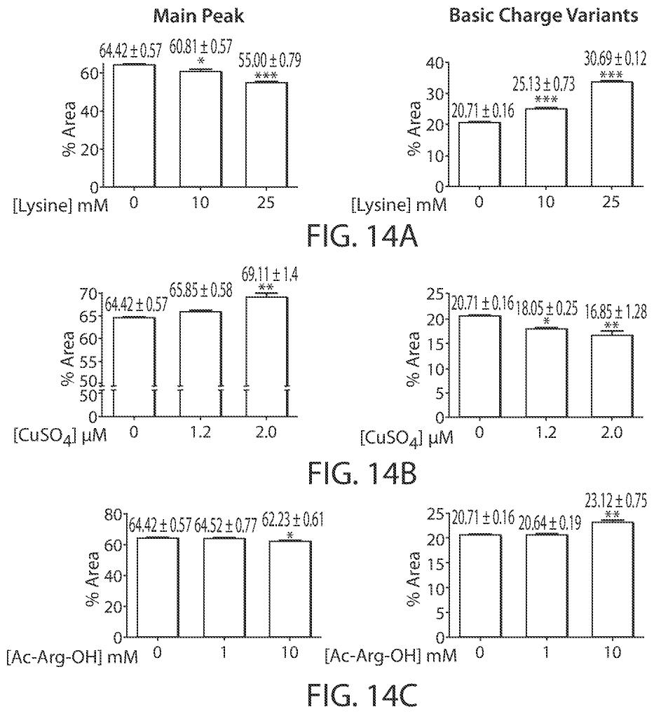

The addition of both 50 mM and 25 mM additional lysine caused a reduction in cell growth as evident by a reduction in the viable cell density (FIGS. 13B-13C). 10 mM additional lysine increased the abundance of the basic peaks (25±0.7%) with a simultaneous decrease in the abundance of the main peak (61±0.6%) when compared to no additional lysine (Basic: 21±0.2%, p<0.001; Main: 64±0.6%, p<0.001; FIG. 14A). Similarly to lysine, N-acetylarginine caused an increase in the basic charge variants, while decreasing the main peak (FIG. 14C). However, the effect of N-acetylarginine was less robust than that of lysine and did not affect cell growth (FIG. 13C) or the metabolic profile. Following supplementation with an additional 2.0 μM CuSO₄ a significant increase in the main peak and significant decrease in the basic peaks were observed (FIG. 15D). The change in the relative amounts of these charge variants were also calculated (FIG. 15E) (see example 4).

From this study, 2.0 M additional CuSO₄ and 10 mM additional lysine were each identified as effective chemical compounds for modulating charge variance in the perfusion reactor.

### Example 4: Charge Variance Modulation by CuSO₄ in a Perfusion Reactor

Following 25 days of growth in a perfusion reactor, 30 mL retains from the perfusate side of the hollow fiber filter were removed daily and stored at −80° C. for protein A purification and subsequent charge variance analysis. Following 5 days of growth under the basal condition (Days 25 to 29), the concentration of CuSO₄ in the reactor was supplemented with an additional 2.0 M CuSO₄ instantaneously through an addition of a nutrient bolus. At the same time, a new media bag containing an additional concentration of 2.0 M CuSO₄ was attached and perfused through the reactor for 4 days. Within a day of introducing the additional 2.0 M CuSO₄, the charge variance profile changed to a new steady-state. This new charge variance state had higher abundance of main peak (72±0.3%) and lower abundance of basic peak (11±0.3%) when compared to the charge variance profile of the basal condition (Main: 68±0.5%, p<0.001; Basic: 14±0.3%, p<0.05). These changes in abundance equate to an increase in main peak relative abundance of 6% and a decrease in basic peaks relative abundance of 14% (FIG. 15E). The relative abundance is the difference between the observed charge variant at higher CuSO₄ concentration and charged variant at basal conditions relative to charge variant at basal conditions expressed as percentage. This experiment demonstrated the ability to produce two different charge variance profiles from a single bioreactor.

### Example 5: Two Charge Variance Modulations in a Perfusion Reactor

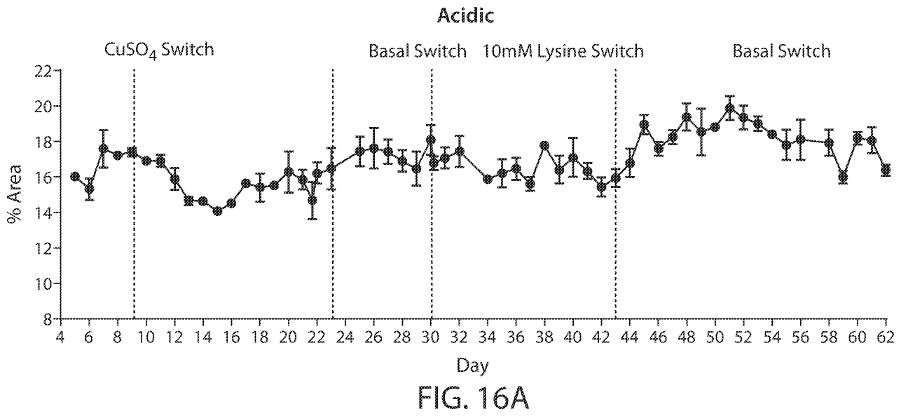

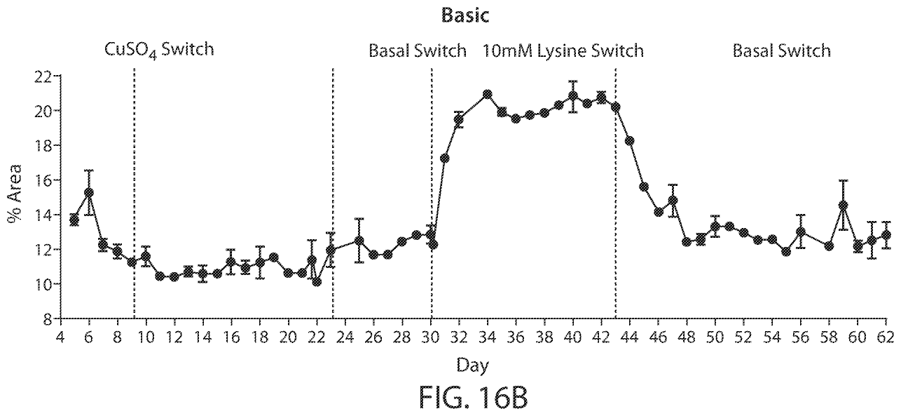

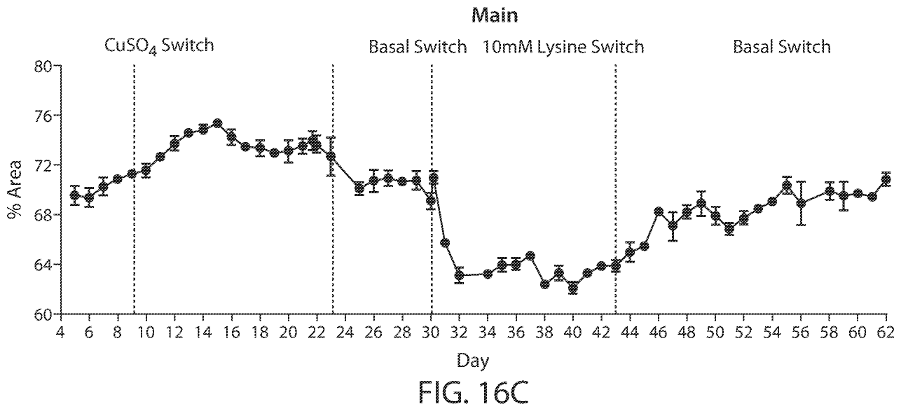

To display the ability to modulate charge variance profiles in opposite directions in a single perfusion reactor, four alternating step-changes were conducted: (i) an increase to 2.0 M additional CuSO₄, (ii) a return to the basal condition, (iii) an increase to 10 mM additional lysine, and (iv) a subsequent return to the basal condition. Over the duration of this experiment, 30 mL samples from the perfusate side of the hollow fiber filter were pulled each day, and the mAb was purified and analyzed for charge variance. Steady-state values, as determined by the leveling off of charge variance, were used to compare the difference in charge variance between the step-changes. FIGS. 16A-16C shows the changes in percent area over time for each of the acidic (FIG. 16A), basic (FIG. 16B), and main (FIG. 16C) peaks. The observed changes are described in detail in subsequent paragraphs below.

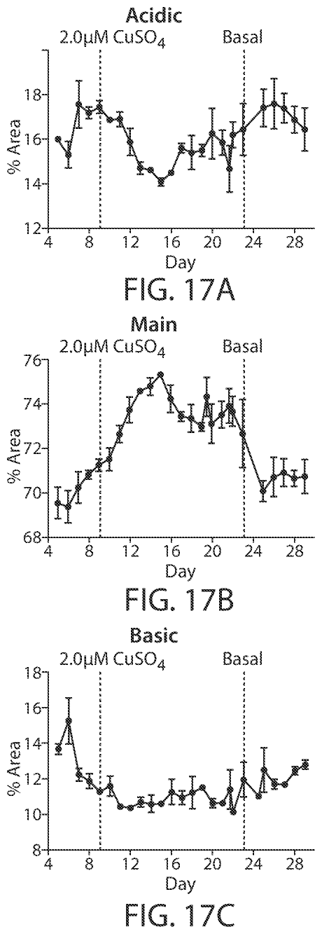

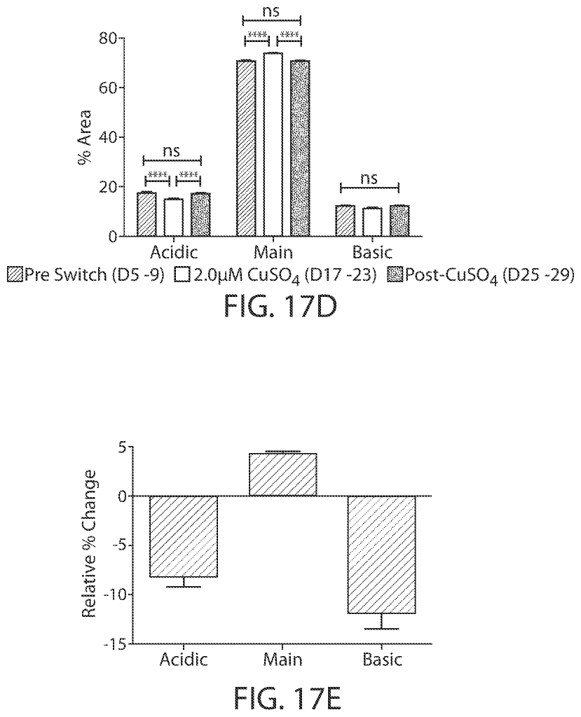

The supplementation of the perfusion reactor with 2.0 M CuSO₄ caused a significant increase in the abundance of the main peak (74±0.2%) when compared to the pre-switch (71±0.5%, p<0.0001) and post-switch steady-state (71±0.2%, p<0.0001) (FIG. 17B). In addition, this change in main peak abundance was accompanied by a significant decrease in the acidic peaks (15±0.3%) when compared to the pre-switch (17±0.6%, p<0.0001) and post-switch steady-states (17±0.2%, p<0.0001) (FIG. 17A). These changes in charge variance equate to an increase in relative abundance of the main peak of 4% and a decrease in relative abundance of the acidic peaks of 8%. No substantial change in the basic peaks was observed (FIG. 17C). This data shows the ability of CuSO₄ to modulate charge variance in a reversible manner, as there was no significant difference between the pre- and post-CuSO₄ steady-state charge variance profiles (FIGS. 17D-17E).

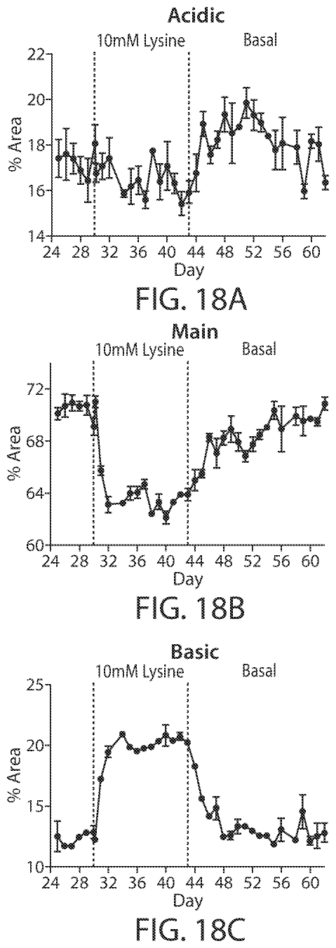

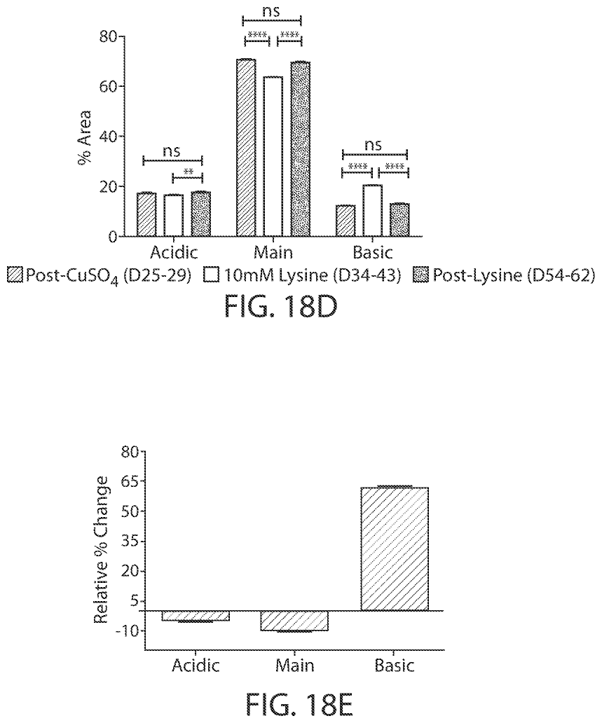

The supplementation of the perfusion reactor with 10 mM lysine caused a significant reduction in the abundance of the main peak (64±0.3%) when compared to the pre-switch steady-state (71±0.2%, p<0.0001) and the post-switch steady state (70±0.2%, p<0.0001) (FIG. 18B). In addition, the abundance of the basic peak was significantly increased following 10 mM lysine supplementation (20±0.1%) when compared to the pre-switch steady-state (12±0.3%, p<0.0001) and post-switch steady-state (13±0.3%, p<0.0001) (FIG. 18C). These changes in charge variance equate to a decrease in relative abundance of the main peak of 10% and increase in the relative abundance of the basic peaks of 62%. These data show that lysine can be used to modulate charge variance in a reversible manner, as there was no significant difference between the pre- and post-lysine steady-state charge variance profiles.

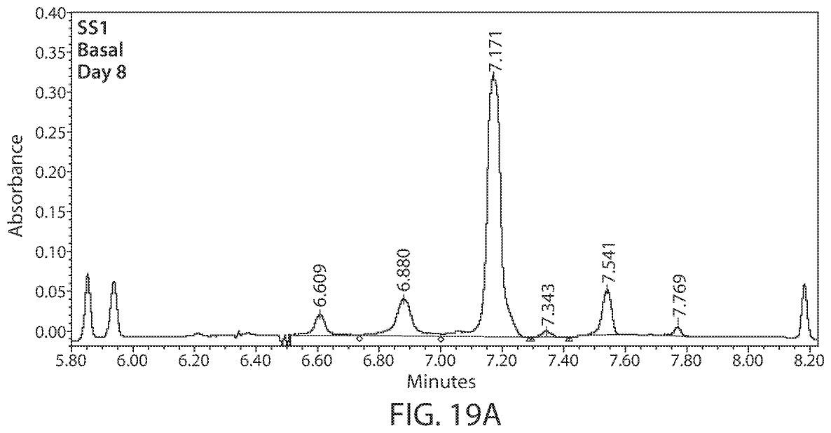

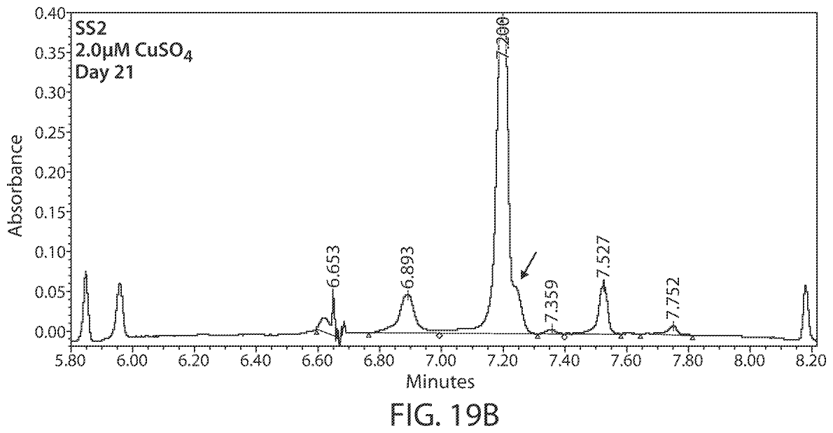

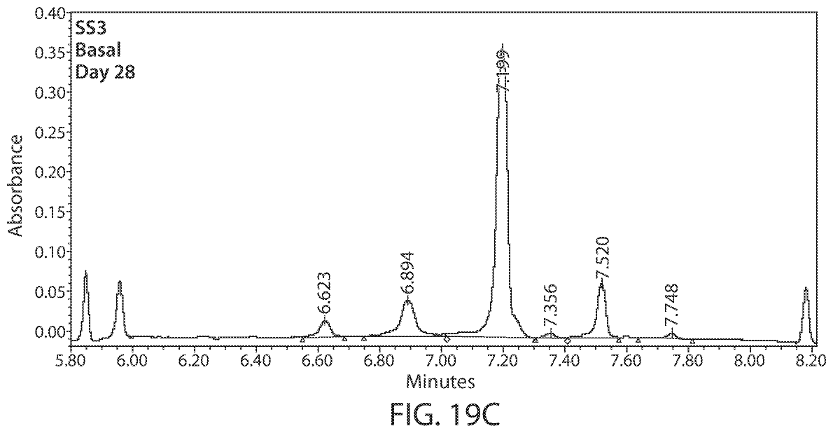

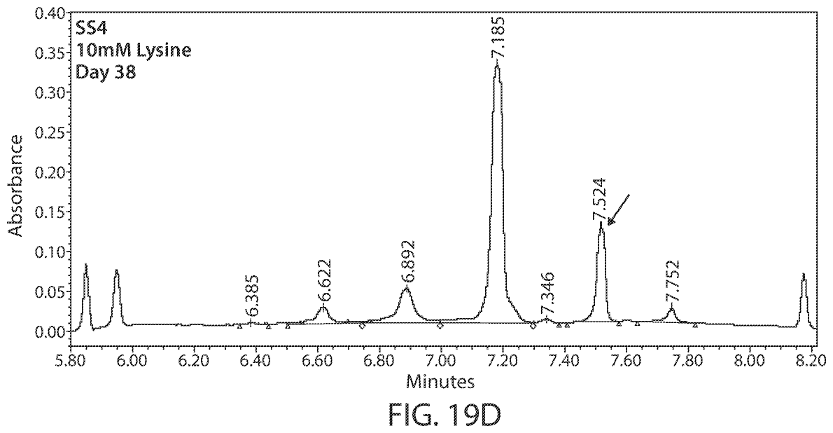

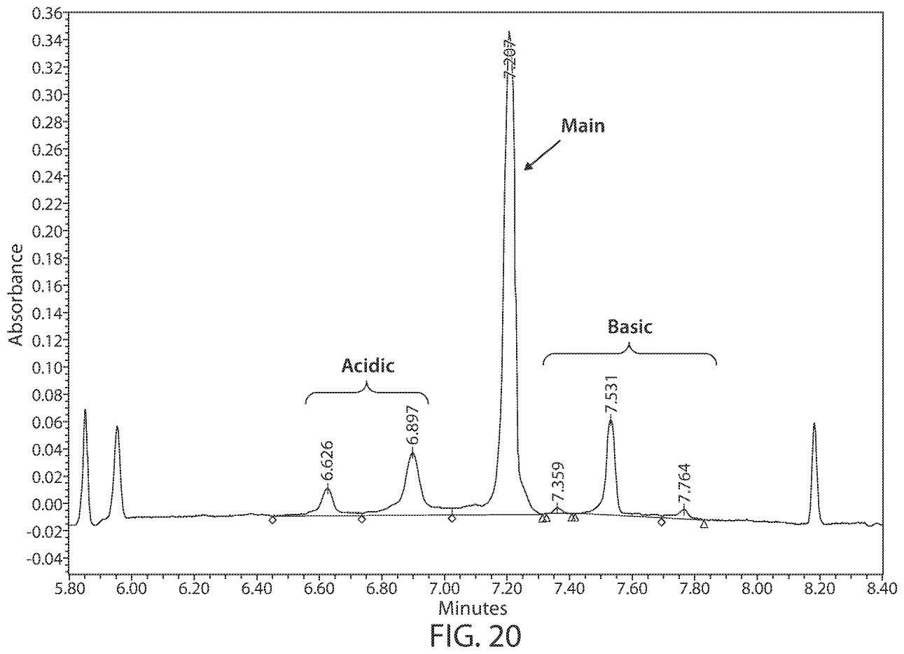

Example electropherograms displaying the differences in charge variance are shown in FIGS. 19A-19D and 20. Generally, the effect of CuSO₄ is evident by the growth of a shoulder on the basic side of the main peak (pI 7.2) (FIG. 19B), while the effect of lysine is seen with a reduction in the main peak (pI 7.2) and growth of the basic peaks, particularly the peak with a pI of 7.5 (FIG. 19D).

Taken together, these data demonstrate manipulation of mAb quality attributes in opposite directions i.e. an increase in abundance of a quality attribute with the addition of chemical 1 as well as decrease in abundance of the same quality attribute with the addition of chemical 2 after removal of chemical 1 in a single bioreactor.

### Example 6: Methods

The methods described in this example were used for the experiments described herein, e.g., in Examples 2-5.

### mAb Purification for Charge Variance Analysis

The produced mAb was purified using protein A spin columns. Briefly, the spin columns were first washed with 600 uL of binding buffer (20 mM Sodium Phosphate, pH 7.0) twice. Then 4.8-6.0 mLs of perfusate was applied to the protein A spin columns in aliquots of 600 uL, for a total of 8-10 applications. Following binding, mAbs were eluted with two additions of 400 uL of elution buffer (0.1M Glycine, 0.1M NaCl, pH 3.5) and neutralized with the addition of 60 uL of 1M Tris, pH 9.0. Subsequently, the concentration of mAbs in each sample was determined using the following HPLC Protein A methodology.

### HPLC Protein A mAb Quantification

mAb quantification was accomplished using a protein A column (0.1 mL, Life Technologies; Bedford, MA). The method consisted of two mobile phases: (i) Binding Buffer (50 mM Glycine, 150 mM NaCl, pH 8.0) and (ii) Elution Buffer (50 mM Glycine, 150 mM NaCl, pH 2.5). All samples were diluted 1:1 in binding buffer prior to injection onto the HPLC. The flow rate of the buffers was constant at 2 mL/min. HPLC analysis for each sample took approximately 2 minutes. The chromatographic method was as follows: for the first 30 seconds, 100% binding buffer was pumped in, followed by 100% elution buffer for 46 seconds, and then re-equilibrated with 100% binding buffer for 14 seconds. Absorbance was measured on a diode array detector at a wavelength of 280 nm. Absorbance was compared to a standard curve of a reference standard preparation of the mAb, and the amount of mAb in the sample was calculated.

### Charge Variance Analysis

150 ug of purified mAb was added to an Amicon Ultra 30 kDa MWCO centrifugal filter (in triplicate) and concentrated down to a volume of 20-30 μL by centrifugation at 14,000 RCF for 20 minutes. This sample was then added to 175 uL of Master Mix solution (0.5% Methylcellulose, 2M Urea, 1% Pharmalytes pH 3-10, 3% Pharmalytes pH 6.7-7.7, and the following the pI markers at 5 L/mL: 5.85, 6.14, 8.18). Each sample was vortexed at high speed for 30 seconds, followed by centrifugation at 9300 RCF for 3 minutes. 150 L of sample was then transferred into 2 mL vials with 300 L inserts and placed into the autosampler of an iCE 3 system set at 8° C. Charge variance was then measured with an initial focus period of 1 minute at 1500V, followed by a final focus period for 11 minutes at 3000V.

### Data Analysis and Statistics

Electropherograms were exported out of the iCE software as Empower files and imported into Empower for analysis. A processing method was designed that integrated the peaks present in the electropherograms and calculated the percent area of each peak. For each of these electropherograms 7 peaks were commonly observed, specifically at pIs of 6.6, 6.9, 7.2, 7.3, 7.5, and 7.7. By area, the charge variant at pI 7.2 was considered the main peak as it represented the variant with the greatest percent area. For analysis the variants with a pI of 6.6 and 6.9 were considered the acidic variants, while the variants at pI 7.3, 7.5, and 7.7 were considered the basic variants (see, e.g., the exemplary electropherogram shown in FIG. 20). For statistical analysis, the sum of these variants's respective percent area were added together to obtain total peak area and to calculate the percent area of each respective group relative to the total peak area. Percent area values were then averaged across steady-states and the means were generally compared using Two-Way ANOVA followed by Bonferroni Post-Hoc Analysis.

### Example 7: Varying Galactosylation Levels

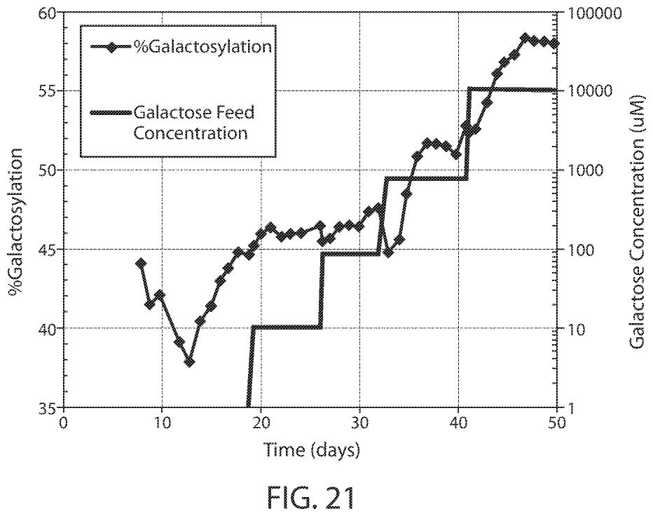

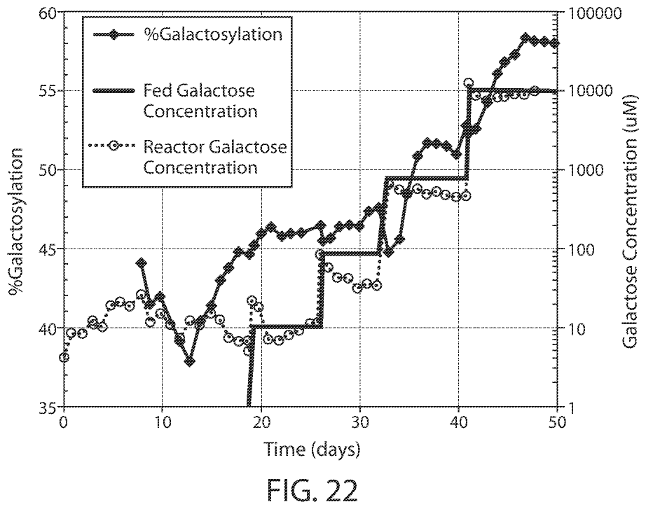

In another example, the perfusion apparatus described above was used to produce multiple fractions of product with different galactosylation levels. It is contemplated that any nutrient that has an effect on galactosylation can be used to influence the galactosylation level of the product. In this example, galactose was used to influence the galactosylation level of the product. Reactor galactose concentration was modulated in the range of 0-10 mM in discrete step increases. The galactosylation of the product in the reactor was allowed to reach a steady-state value before proceeding to the next step change. The galactose concentrations fed into the reactor were 0, 0.01, 0.086, 0.79, and 10 mM. Corresponding steady-state galactosylation values of 46%, 46%, 46%, 52%, and 58% were achieved. FIG. 21 shows the step increases in galactose concentration and the corresponding changes in product percent galactosylation over time in the perfusion apparatus. FIG. 22 further overlays the reactor galactose concentration detected at the indicated time points. See, e.g., Downey et al., Biotechnol Prog. 2017 November; 33(6):1647-1661, which is incorporated herein by reference in its entirety.

1-65. (canceled)

66. A method of making a plurality of protein variants by a perfusion production culture, the plurality comprising at least protein variant 1 and protein variant 2, in a single vessel which is operated continuously through production of the protein variant 1 and the protein variant 2, comprising:
(a) culturing a population of cells in culture medium in the vessel under a first condition to form a first variant preparation containing the protein variant 1;
(b) recovering the first variant preparation;
(c) purifying the protein variant 1 from the first variant preparation;
(d) measuring one or more target parameters selected from: amount of the protein variant 1 produced, duration of culture under the first condition, or viability of culture;
(e) once the one or more target parameters has reached a target value, culturing the population of cells in culture medium in the vessel under a second condition to form a second variant preparation comprising the protein variant 2;
(f) recovering the second variant preparation; and
(g) purifying the protein variant 2 from the second variant preparation;
thereby providing a plurality of purified protein variants, the plurality comprising at least purified protein variant 1 and purified protein variant 2,
wherein the protein variant 1 differs from the protein variant 2 by a physical, chemical, biological, or pharmaceutical property.

67. The method of claim 66, further comprising culturing the population of cells under the first condition after step (b) to form additional first variant preparation.

68. The method of claim 66, further comprising culturing the population of cells under the second condition after step (f) to form additional second variant preparation.

69. The method of claim 66, comprising manipulation of the medium to achieve the second condition.

70. The method of claim 69, wherein manipulation of the medium comprises adding a concentrated bolus of one or more of the following to the culture medium: lysine, galactose, a water soluble copper compound, or N-acetylarginine.

71. The method of claim 69, wherein manipulation of the medium comprises increasing the concentration of one or more of the following in the culture medium entering the vessel: lysine, galactose, a water soluble copper compound, or N-acetylarginine.

72. The method of claim 69, wherein manipulation of the medium comprises increasing or decreasing the concentration in the medium inside the vessel of CuSO4; lysine; N-acetylarginine and/or galactose.

73. The method of claim 66, wherein the protein variant 1 differs from the protein variant 2, by one or more of: glycosylation, galactosylation, sialylation, charge, pI, N-terminal or C-terminal sequence, homogeneity, deamidation, glycation, proline amidation, disulfide heterogeneity, dimerization, and methionine oxidation.

74. The method of claim 66, further comprising:
(g) culturing the population of cells in culture medium in the vessel under a third condition to form a third variant preparation containing protein variant 3; and
(h) recovering the third variant preparation, and
(i) purifying the protein variant 3 from the third variant preparation.

75. The method of claim 66, wherein the vessel is a bioreactor.

76. The method of claim 66, wherein the amount removed during recovering step (b) is between 0.1 to 5 times the vessel volume per day of vessel operation.

77. The method of claim 66, wherein the first and/or second conditions are steady state conditions.

78. A preparation of a plurality of variant products described herein or, made by, or makeable by, the method of claim 66.

79. A pharmaceutical composition comprising the preparation of claim 78.

80. The pharmaceutical composition of claim 79, comprising a pharmaceutically acceptable diluent, carrier, or excipient.

81. A kit comprising a plurality of variant products of claim 78.

82. The kit of claim 81, wherein each variant product of the plurality is separately packaged into containers.

83. The kit of claim 82, wherein at least one container comprises product variant 1 that is at least 50% product variant 1.

84. The kit of claim 82, wherein at least one container comprises product variant 2 that is at least 50% product variant 2.
